(19) United States
(12) Patent Application Publication
(10) Pub. No.: US 2024/0408204 A1
(43) Pub. Date: Dec. 12, 2024

(54) Methods of Treating Lymphoma with a Phagocyte Having a Chimeric Antigen Receptor

(71) Applicant: The Regents of the University of California, Oakland, CA (US)

(72) Inventor: Denise Montell, Santa Barbara, CA (US)

(21) Appl. No.: 18/702,635

(22) PCT Filed: Oct. 27, 2022
(86) PCT No.: PCT/US2022/078774

§ 371 Date: Apr. 18, 2024

Related U.S. Application Data
(60) Provisional application No. 63/273,109, filed on Oct. 28, 2021.

(51) Int. Cl. A61K 39/00; C07K 14/725; C07K 16/28; C12N 9/14

(52) U.S. Cl. CPC A61K 39/4614; A61K 39/4631; A61K 39/464412; C07K 14/7051; C07K 16/2803; C12N 9/14; C12Y 306/01

(57) ABSTRACT

Compositions and methods are provided for treating lymphoma in an individual by targeting engineered phagocytes to lymphoma cells. In particular, the phagocytes provided for administration to an individual, who has lymphoma, are engineered to express a chimeric antigen receptor (CAR) that specifically binds to an antigen present on lymphoma cells. The CAR localizes the engineered phagocytes to sites where lymphoma cells are present. In some embodiments, phagocytic activity of the phagocytes is enhanced by further engineering the phagocytes to express a hyperactive Rac GTPase.

### CROSS REFERENCE TO RELATED APPLICATIONS

This application claims benefit of U.S. Provisional Patent Application No. 63/273,109, filed Oct. 28, 2021, which application is incorporated herein by reference in its entirety.

### INCORPORATION BY REFERENCE OF A SEQUENCE LISTING

A Sequence Listing is provided herewith as a Sequence Listing XML file, “UCSB-549WO”, created on Oct. 21, 2022, and having a size of 18,352 bytes. The contents of the Sequence Listing XML file are incorporated by reference herein in their entirety.

### BACKGROUND OF THE INVENTION

Various methods and systems have been developed to target cells in a manner that results in death of a targeted cell. Cellular immunotherapies using chimeric antigen receptor T-cells (CAR-T) are revolutionizing cancer treatment. In CAR-T therapy, a patient's T cells are removed, engineered to express a chimeric antigen receptor (CAR) that binds to a tumor antigen, expanded ex vivo and reinfused into the patient. Without question, CAR-T has prolonged lives; however, there is enormous room for improvement because CAR-T therapy is hampered by a number of limitations, including: 1) CAR-T-cells frequently fail to infiltrate into tumors; 2) CAR-T cells become exhausted; 3) tumor cells lacking the target antigen escape; 4) cytokine storms and auto-immune reactions interfere, and 5) the therapy is complex, expensive, and time-consuming.

Although progress has been made using chimeric antigen receptors, particularly to reprogram T cells to target and kill cancerous cells, challenges still remain for developing methods of using chimeric antigen receptors with other immune cells for treatment of a disease.

### SUMMARY OF THE INVENTION

Compositions and methods are provided for treating lymphoma in an individual by targeting engineered phagocytes to lymphoma cells. In particular, the phagocytes provided for administration to an individual, who has lymphoma, are engineered to express a chimeric antigen receptor (CAR) that specifically binds to an antigen present on lymphoma cells. The CAR localizes the engineered phagocytes to sites where lymphoma cells are present. In some embodiments, phagocytic activity of the phagocytes is enhanced by further engineering the phagocytes to express a hyperactive Rac GTPase.

In one aspect, a composition comprising a phagocyte specific for lymphoma cells is provided, wherein the phagocyte is engineered to express a chimeric antigen receptor that specifically binds to a target lymphoma antigen, wherein the phagocyte is further engineered to express a hyperactive Rac GTPase.

In certain embodiments, the composition further comprises a pharmaceutically acceptable excipient.

In certain embodiments, the phagocyte is a macrophage, a monocyte, a neutrophil, or a dendritic cell, or a precursor thereof.

In certain embodiments, the expression of the hyperactive Rac GTPase in the phagocyte increases phagocytic activity of the phagocyte.

In certain embodiments, the Rac GTPase is Rac family small GTPase 2 (RAC2). In some embodiments, the RAC2 comprises a substitution of lysine for glutamic acid at amino acid position 62, wherein numbering of amino acid positions is relative to the reference amino acid sequence of SEQ ID NO:1.

In certain embodiments, the chimeric antigen receptor comprises a transmembrane domain linked to an extracellular antigen binding domain that binds specifically to the target lymphoma antigen and an intracellular engulfment signaling domain, wherein the transmembrane domain is positioned between the extracellular antigen binding domain and the intracellular engulfment signaling domain. In some embodiments, the extracellular antigen binding domain comprises a single chain variable fragment (scFv) that binds specifically to the target lymphoma antigen. In some embodiments, the transmembrane domain is a CD8 transmembrane domain. In some embodiments, the chimeric antigen receptor further comprises an extracellular spacer domain positioned between and connecting the extracellular antigen binding domain and the transmembrane domain. In some embodiments, the chimeric antigen receptor further comprises an intracellular spacer domain positioned between and connecting the intracellular engulfment signaling domain and the transmembrane domain.

Upon binding of the extracellular antigen binding domain to the target lymphoma antigen, the engulfment signaling domain stimulates engulfment signaling activity that directs the engineered phagocyte to engulf the lymphoma cell. In some embodiments, the engulfment signaling domain is a homeostatic engulfment signaling domain. Exemplary homeostatic engulfment signaling domains include, without limitation, MRC1, ItgB5, MERTK, Tyro3, and Axl signaling domains. In other embodiments, the engulfment signaling domain is a pro-inflammatory engulfment signaling domain. Exemplary pro-inflammatory engulfment signaling domains include, without limitation, Traf6, Syk, MyD88, Zap70, FcγR1, FcγR2A, FcγR2B2, FcγR2C, FcγR3A, FcεR1, FcαR1, BAFF-R, NFAM1, DAP12, and CD79b signaling domains. In still other embodiments, the engulfment signaling domain includes a primary engulfment signaling domain and a secondary engulfment signaling domain. In such embodiments, the primary engulfment signaling domain and the secondary engulfment signaling domain can be independently selected from homeostatic and pro-inflammatory engulfment signaling domains, including those described herein.

In certain embodiments, the target lymphoma antigen is selected from the group consisting of CD2, CD3, CD4, CD5, CD10, CD15, CD19, CD20, CD22, CD30, CD43, CD79a, BCL-2, BCL6, Pax-5, TdT, LCA, Oct-2, BOB.1, Ki67, and Epstein-Barr virus-latent membrane protein (EBV-LMP).

In certain embodiments, the lymphoma is Burkitt lymphoma.

In certain embodiments, the hyperactive Rac GTPase is expressed as a fusion protein linked to the chimeric antigen receptor by a cleavable peptide.

In certain embodiments, the hyperactive Rac GTPase is expressed independently of the chimeric antigen receptor.

In certain embodiments, the phagocyte is engineered to express the chimeric antigen receptor and the hyperactive Rac GTPase by transfecting the phagocyte with a first recombinant polynucleotide encoding the chimeric antigen receptor and a second recombinant polynucleotide encoding the hyperactive Rac GTPase or transfecting the phagocyte with a bicistronic recombinant polynucleotide encoding the chimeric antigen receptor and the hyperactive Rac GTPase. In some embodiments, the first recombinant polynucleotide, the second recombinant polynucleotide, or the bicistronic recombinant polynucleotide is provided by a plasmid, a viral vector, or a messenger RNA. In some embodiments, the bicistronic recombinant polynucleotide comprises an internal ribosome entry site (IRES) or a 2A element. In some embodiments, the first recombinant polynucleotide comprises a promoter operably linked to a nucleotide sequence encoding the chimeric antigen receptor. In some embodiments, the second recombinant polynucleotide comprises a promoter operably linked to a nucleotide sequence encoding the hyperactive Rac GTPase. In some embodiments, the second recombinant polynucleotide comprises a promoter operably linked to a first nucleotide sequence encoding the chimeric antigen receptor and a second nucleotide sequence encoding the hyperactive Rac GTPase. In some embodiments, the promoter is constitutive or inducible. In some embodiments, the promoter is a phagocyte promoter.

In certain embodiments, the phagocyte is associated with all or a portion of a lymphoma cell, wherein the chimeric antigen receptor specifically binds to the target lymphoma antigen on the lymphoma cell. In some embodiments, the lymphoma cell is partially or fully engulfed by the engineered phagocyte.

In certain embodiments, the composition further comprises an anti-cancer therapeutic agent. In some embodiments, the anti-cancer therapeutic agent is selected from the group consisting of a chemotherapeutic agent, an immunotherapeutic agent, a biologic therapeutic agent, a pro-apoptotic agent, an angiogenesis inhibitor, a photoactive agent, a radiosensitizing agent, and a radioisotope.

In certain embodiments, a composition comprising a phagocyte specific for lymphoma cells for use in a method of treating lymphoma is provided, wherein the phagocyte is engineered to express a chimeric antigen receptor that specifically binds to a target lymphoma antigen, wherein the phagocyte is further engineered to express a hyperactive Rac GTPase

In another aspect, a method of treating lymphoma in a subject in need thereof is provided, the method comprising administering to the subject a therapeutically effective amount of a composition comprising an engineered phagocyte, described herein.

In certain embodiments, the phagocyte has anti-tumor activity.

In certain embodiments, the expression of the Rac GTPase is inducible.

In certain embodiments, the phagocyte is autologous or allogeneic.

In certain embodiments, the phagocyte is engineered and expanded in culture prior to said administering.

In certain embodiments, the subject is a human.

In certain embodiments, the method further comprises administering an anti-cancer therapeutic agent. In some embodiments, the anti-cancer therapeutic agent is selected from the group consisting of a chemotherapeutic agent, an immunotherapeutic agent, a biologic therapeutic agent, a pro-apoptotic agent, an angiogenesis inhibitor, a photoactive agent, a radiosensitizing agent, and a radioisotope. Exemplary chemotherapeutic agents include, without limitation, methotrexate, cyclophosphamide, doxorubicin, vincristine, cytarabine, ifosfamide, and etoposide. Exemplary immunotherapeutic agent include, without limitation, rituximab.

In certain embodiments, the method further comprises administering a steroid. Exemplary steroids include, without limitation, prednisolone.

In certain embodiments, multiple therapeutically effective doses of the phagocyte are administered to the subject. In some embodiments, multiple cycles of treatment are administered to the subject for a time period sufficient to effect at least a partial tumor response. For example, multiple cycles of treatment may be administered for at least 2 months, at least 4 months, at least 6 months, at least 12 months, or longer. In some embodiments, multiple therapeutically effective doses of the phagocyte are administered to the subject until a complete tumor response is effected. In some embodiments, treatment results in a reduction in tumor size, a reduction in the number of lymphoma cells, slowing or halting of tumor growth, slowing or halting of cancer cell infiltration into peripheral organs, slowing or halting of tumor metastasis, or a combination thereof.

In certain embodiments, the lymphoma is Burkitt lymphoma.

In another aspect, a kit comprising a composition comprising an engineered phagocyte, described herein, and instructions for treating lymphoma are provided.

In another aspect, a method of increasing phagocytosis or trogocytosis of a lymphoma cell in a subject is provided, the method comprising administering to the subject an effective amount of an engineered phagocyte, wherein the engineered phagocyte is engineered to express a chimeric antigen receptor that specifically binds to a target lymphoma antigen on the lymphoma cell, wherein the engineered phagocyte is further engineered to express a hyperactive Rac GTPase, wherein the lymphoma cell undergoes phagocytosis or trogocytosis by the engineered phagocyte at an increased rate compared to the rate of phagocytosis or trogocytosis of a control phagocyte that is not engineered to express the chimeric antigen receptor and the hyperactive Rac GTPase. In some embodiments, the lymphoma cell is wholly or partially engulfed by the engineered phagocyte.

In another aspect, a method of treating lymphoma in a subject in need thereof is provided, the method comprising administering one or more expression vectors comprising a first expression cassette comprising a coding sequence encoding a chimeric antigen receptor that specifically binds to a target lymphoma antigen and a second expression cassette comprising a coding sequence encoding a hyperactive RAC2 GTPase, wherein the chimeric antigen receptor and the hyperactive RAC2 GTPase are expressed in vivo in a phagocyte in the subject in a therapeutically effective amount sufficient to result in phagocytosis or trogocytosis of a lymphoma cell.

In certain embodiments, the first expression cassette and the second expression cassette are in separate vectors.

In certain embodiments, the first expression cassette and the second expression cassette are in a bicistronic vector comprising an internal ribosome entry site (IRES) or a 2A element.

In certain embodiments, the first expression cassette comprises a promoter operably linked to the coding sequence encoding the chimeric antigen receptor. In some embodiments, the promoter is a constitutive or an inducible promoter. In some embodiments, the promoter is a phagocyte promoter. In certain embodiments, the second expression cassette comprises a promoter operably linked to the coding sequence encoding the hyperactive RAC2 GTPase. In certain embodiments, the promoter is a constitutive or inducible promoter. In certain embodiments, the promoter is a phagocyte promoter.

In another aspect, a composition comprising one or more expression vectors comprising a first expression cassette comprising a coding sequence encoding a chimeric antigen receptor that specifically binds to a target lymphoma antigen and a second expression cassette comprising a coding sequence encoding a hyperactive RAC2 GTPase is provided, wherein the chimeric antigen receptor and the RAC2 GTPase are expressed in vivo in a phagocyte in the subject in a therapeutically effective amount sufficient to result in phagocytosis or trogocytosis of a lymphoma cell.

In certain embodiments, a composition comprising one or more expression vectors comprising a first expression cassette comprising a coding sequence encoding a chimeric antigen receptor that specifically binds to a target lymphoma antigen and a second expression cassette comprising a coding sequence encoding a hyperactive RAC2 GTPase for use in a method of treating lymphoma is provided, wherein the chimeric antigen receptor and the RAC2 GTPase are expressed in vivo in a phagocyte in the subject in a therapeutically effective amount sufficient to result in phagocytosis or trogocytosis of a lymphoma cell.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. Race CAR-P (Rac-enhanced CAR-Phagocyte) immunotherapy. (1) An antigen on target cells binds to a CAR expressed by a phagocyte (2). Co-expression of an active form of Rac (3) is sufficient to greatly enhance engulfment by CAR-expressing macrophages (4a). Rac especially enhances their ability to eat and destroy multiple, whole, living target cells. Rac also stimulates inflammatory cytokines (4b, 5b). Engulfment is expected to debulk the tumor by killing the target (5a) and result in antigen presentation (6a and 7a) and simulation of an adaptive immune response (6b).

FIGS. 2A-2C. Border cells expressing active Rac kill egg chambers by eating nurse cells alive. (FIG. 2A) slboGal4, UAS-GFP expression in border cells. (FIG. 2B) co-expression of active Rac results in egg chamber destruction. (FIG. 2C) mutating the Draper (Qrru:) receptor rescues egg chamber viability.

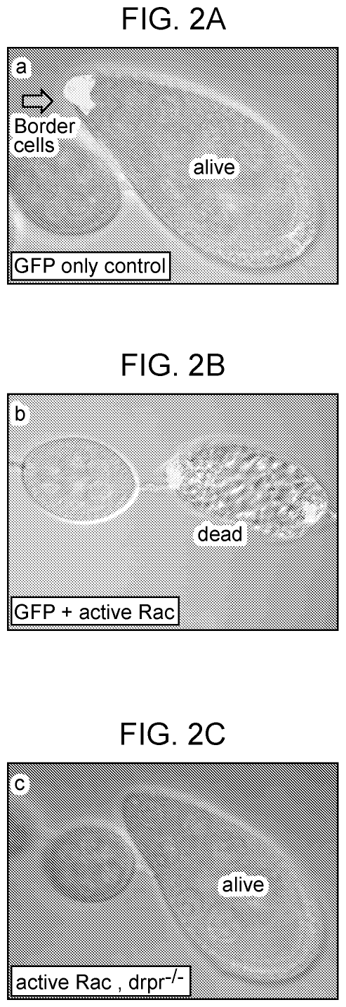

FIG. 3. Polar cell engulfment by Rac^{G12V} border cell. Confocal images of forming border cell clusters in slboGal4; UAS-PHdomain-GFP egg chambers. p-polar cell, b-border cell, *engulfed polar cell.

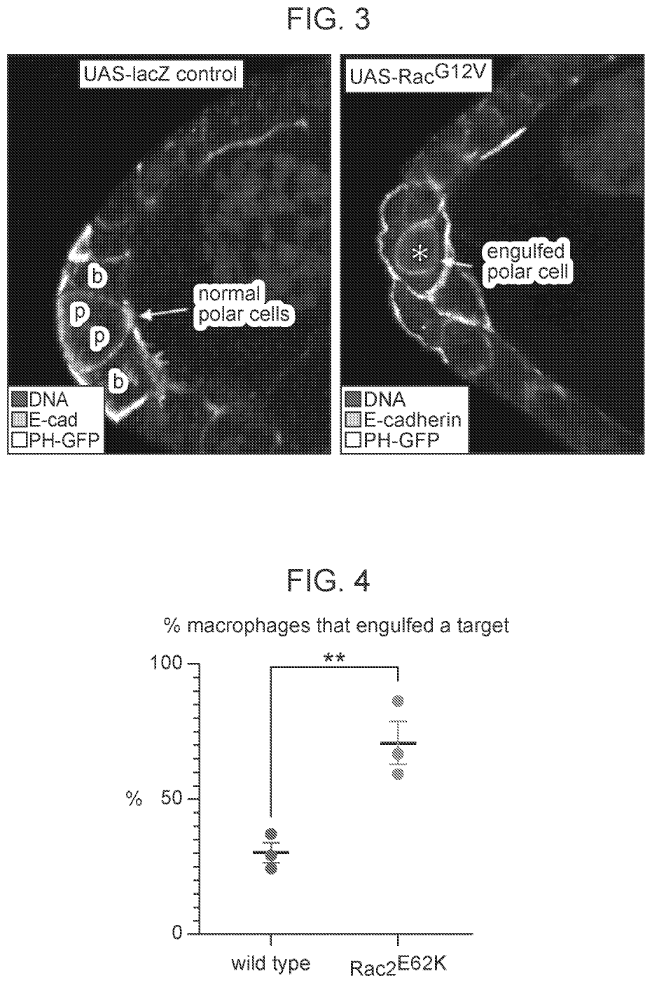

FIG. 4. RAC2^{E62KI+} bone marrow-derived macrophages engulf and kill more T cells than wild type macrophages do.

FIG. 5. RAC2^{E62K}-expressing human macrophages engulf and kill more human Jurkat T leukemia cells than control macrophages. Asterisks mark engulfed cells.

FIGS. 6A-6D. Anti-CD19 Race CAR-macrophages specifically and avidly engulf CD19-positive Raji B cell lymphoma cells. Representative still images from a time lapse movie of macrophages of genotypes: no CAR, Rac^{+/+} (FIG. 6A), no CAR, Rac^{E62K/+} (FIG. 6B), anti-CD19 CAR, Rac^{+/+} (FIG. 6C), and anti-CD19 CAR, Rac^{E62K/+} (FIG. 6D) as they interact with target cells. Note that in the absence of the appropriate CAR, RAC2^{E62K} macrophages do not engulf the targets, showing that RAC2^{E62K} enhances only engulfment events triggered by specific ligand/receptor binding.

### DETAILED DESCRIPTION

Compositions and methods are provided for treating lymphoma in an individual by targeting engineered phagocytes to lymphoma cells. In particular, the phagocytes provided for administration to an individual, who has lymphoma, are engineered to express a chimeric antigen receptor (CAR) that specifically binds to an antigen present on lymphoma cells. The CAR localizes the engineered phagocytes to sites where lymphoma cells are present. In some embodiments, phagocytic activity of the phagocytes is enhanced by further engineering the phagocytes to express a hyperactive Rac GTPase.

### Definitions

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the protein” includes reference to one or more proteins and equivalents thereof, e.g., polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

General methods in molecular and cellular biochemistry can be found in such standard textbooks as Molecular Cloning: A Laboratory Manual, 3rd Ed. (Sambrook et al., CSH Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

The term “about”, as used herein, has its ordinary meaning of approximately. As such, the term “about” is used to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The terms “phagocytic cell” or “phagocyte” or plural forms thereof as used herein indicate a cell that is capable of phagocytosis, which is the process by which a cell uses its plasma membrane to engulf a particle giving rise to an internal compartment called the phagosome. Phagocytosis is one type of endocytosis as will be understood by a skilled person. Phagocytes of an individual typically use their plasma membrane to engulf and remove cellular debris, foreign substances, microbes, and cells to protect the body of an individual. Phagocytes may also perform trogocytosis or partial phagocytosis, wherein the phagocyte “nibbles” at a target cell rather than completely engulfing the target cell, which may result in the transfer of plasma membrane fragments from the target cell to the phagocyte. In addition, a phagocyte may extract surface molecules from the plasma membrane of a target cell and in some cases a portion of the cytoplasmic contents of the target cell. In some cases, a phagocyte may perform trogocytosis repeatedly until the target cell disintegrates. Phagocytes may kill target cells either by phagocytosis or trogocytosis. Phagocytes include, without limitation, macrophages, monocytes, neutrophils, and dendritic cells.

The term “precursor” or “precursor cell” when used in connection with phagocytes such as macrophages, monocytes, neutrophils, and dendritic cells refers to a parent cell in a cellular lineage that leads to generation of phagocytic cells. Exemplary precursor cells include bone marrow, monoblasts, hematopoietic stem cells, and other precursor cells identifiable by a person skilled in the art.

Phagocytes of various embodiments in accordance with the present disclosure are naturally occurring or engineered phagocytes capable of expressing a chimeric antigen receptor, as described herein, and a hyperactive RAC2 protein and/or a RAC2 protein at an elevated expression level.

The terms “engineered” or “recombinant” in reference to a phagocyte, gene, nucleic acid and/or protein as used herein, refer to a phagocyte, gene, nucleic acid and/or protein that has been altered through human intervention. Accordingly, the term “naturally occurring” as used herein in reference to a phagocyte, gene, nucleic acid and/or protein as used herein, refer to a phagocyte, gene, nucleic acid and/or protein existing in nature and without any human intervention. Exemplary human interventions comprise transfection with a heterologous polynucleotide, molecular cloning resulting in a deletion, insertion, modification and/or rearrangement with respect to a naturally occurring sequence such as a naturally occurring sequence in a phagocyte, gene, nucleic acid and/or protein herein described.

The term “lymphoma” refers to any disease associated with blood and lymph tumors at any stage (e.g., stage I, stage II, stage Ill, or stage IV according to the Ann Arbor staging classification scheme). The term lymphoma includes, but is not limited to, Hodgkin lymphoma, including classic Hodgkin lymphoma and nodular lymphocyte predominant Hodgkin lymphoma; and non-Hodgkin lymphoma, including B-cell lymphomas (e.g., Burkitt lymphoma, diffuse large B-cell lymphoma, follicular lymphoma, chronic lymphocytic leukemia, mantle cell lymphoma, marginal zone lymphoma, and lymphoplasmacytic lymphoma), T-cell lymphomas (e.g., adult T-cell lymphoma, cutaneous T-cell lymphoma, peripheral T-cell lymphoma, anaplastic large cell lymphoma, angioimmunoblastic T-cell lymphoma, adult T-cell leukemia/lymphoma, and lymphoblastic lymphoma), extranodal NK-T-cell lymphoma, gastric lymphoma, primary effusion lymphoma, splenic marginal zone lymphoma, lymphoplasmacytic lymphoma, and multicentric Castleman disease. The term lymphoma also includes any type of lymphoma associated with an infection by Epstein-Barr virus, human T-cell leukemia virus, Helicobacter pylori, human gammaherpesvirus 8, hepatitis C virus, or human immunodeficiency virus.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease.

A “therapeutically effective amount” is intended for an amount of active agent which is necessary to impart therapeutic benefit to a subject. For example, a “therapeutically effective amount” is an amount which induces, ameliorates or otherwise causes an improvement in the pathological symptoms, disease progression or physiological conditions associated with a disease or which improves resistance to a disorder.

By “anti-tumor activity” is intended a decrease in tumor volume, a decrease in the number of tumor cells, a decrease in the number of metastases, an increase in life expectancy, or amelioration of various physiological symptoms associated with a cancerous condition. An “anti-tumor effect” can also be manifested by prevention of a hematological malignancy or tumor formation.

The term “tumor response” as used herein means a reduction or elimination of all measurable lesions. The criteria for tumor response are based on the WHO Reporting Criteria [WHO Offset Publication, 48-World Health Organization, Geneva, Switzerland, (1979)]. Ideally, all uni- or bidimensionally measurable lesions should be measured at each assessment. When multiple lesions are present in any organ, such measurements may not be possible and, under such circumstances, up to 6 representative lesions should be selected, if available.

The term “complete response” (CR) as used herein means a complete disappearance of all clinically detectable malignant disease, determined by 2 assessments at least 4 weeks apart.

The term “partial response” (PR) as used herein means a 50% or greater reduction from baseline in the sum of the products of the longest perpendicular diameters of all measurable disease without progression of evaluable disease and without evidence of any new lesions as determined by at least two consecutive assessments at least four weeks apart. Assessments should show a partial decrease in the size of lytic lesions, recalcifications of lytic lesions, or decreased density of blastic lesions.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer. Both full-length proteins and fragments thereof are encompassed by the definition. The terms also include postexpression modifications of the polypeptide, for example, phosphorylation, glycosylation, acetylation, hydroxylation, oxidation, and the like.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), and any other type of polynucleotide which is an N- or C-glycoside of a purine or pyrimidine base. There is no intended distinction in length between the terms “polynucleotide,” “oligonucleotide,” “nucleic acid” and “nucleic acid molecule,” and these terms are used interchangeably.

By “isolated” is meant, when referring to a protein, polypeptide, or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

As used herein, the terms “increase”, “increasing”, “enhance”, and “enhancing” (and grammatical variations thereof) describes unless the context indicates otherwise a detectable elevation of a reference value. An increase can comprise an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

The term “hyperactive or “activated”, as used herein with reference to a Rac protein, refers to a Rac protein having a modification to its sequence resulting in increased Rac biological activity. One or more Rac biological activities may be enhanced by a mutation, including GTP binding, GTP hydrolysis and/or association with downstream effectors, which mediate Rac effects on various biological events, such as structural changes to the actin, cytoskeletal reorganization, cell growth, cell movement, translocation of glucose transforming vesicles, glucose uptake, antimicrobial cytotoxicity, the activation of protein kinases and additional events identifiable by a skilled person. Exemplary downstream effectors include, without limitation, the serine/threonine-protein kinase, Akt, also known as protein kinase B, as well as serine/threonine protein kinase, p65^{PAK}, also known as PAK1, and additional downstream effectors identifiable by a skilled person.

A hyperactive Rac protein may have one or more mutations that enhance one or more biological activities of a Rac protein. In some embodiments, the hyperactive Rac protein has a mutation (e.g., a point mutation) at one or more amino acid residues corresponding to amino acid residue number 11, 12, 28, 29, 30, 34, 62, 63, 92, and/or 157, wherein numbering of amino acid positions is relative to the reference amino acid sequence of SEQ ID NO 1. In some embodiments, a hyperactive Rac comprises one or more activating mutations selected from D11A, G12V/R, F28L, P29S, P29L, P29Q, PG(29,30)VD, P34H, E62K, D63V N92S, N92T N921, C157Y, wherein numbering of amino acid positions is relative to the reference sequence of SEQ ID NO 1. For a description of activating mutations that increase activity of Rac proteins, see, e.g., International Patent Application No. WO 2021217087 A1, herein incorporated by reference in its entirety.

A RAC2 polynucleotide, nucleic acid, oligonucleotide, protein, polypeptide, or peptide refers to a molecule derived from any source. The molecule need not be physically derived from an organism, but may be synthetically or recombinantly produced. A number of RAC2 nucleic acid and protein sequences are known. A representative human RAC2 amino acid sequence is presented in SEQ ID NO:1 and additional representative sequences are listed in the National Center for Biotechnology Information (NCBI) database. See, for example, NCBI entries for human RAC2 (NM_002872.3 and NP 0028630.1), mouse RAC2 (NM_009008.3 and NP 033034.1), rat RAC2 (NM_001008384.1 and NP_001008385.1), chimpanzee RAC2 (XM_001145815.3 and XP_001145815.3), monkey RAC2 (XM_001086228.2 and XP_001086228.1), dog RAC2 (XM_538392.4 and XP 538392.4), cow RAC2 (NM_I 75792.2 and NP 786986.1), chicken RAC2 (NM_001201452.1 and NP 001188381.1), and zebrafish RAC2 (NM_001002061.1 and NP 001002061.1); all of which sequences (as entered by the date of filing of this application) are herein incorporated by reference. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to construct a hyperactive RAC2 protein comprising a mutation that increases activity (e.g., E62K) or a nucleic acid encoding it, as described herein. Additional, examples of Rac genes and protein sequences can be found in public gene databases such as NCBI, Uniprot and other public genomic and protein sequence databases identifiable to a person skilled in the art. A hyperactive Rac2 has increased biological activity compared to the wild-type Rac2 protein having the amino acid sequence of SEQ ID NO: 1.

As used herein, “reference sequence” is a defined sequence used as a basis for sequence comparison. A reference sequence may be a subset or the entirety of a specified sequence; for example, as a segment of a full-length protein or protein fragment. A reference sequence can comprise, for example, a sequence identifiable in a database such as GenBank and UniProt and others identifiable to those skilled in the art.

Algorithms and programs for comparing primary biological sequence information between any two sequences are identifiable by a skilled person. Non-limiting examples of such mathematical algorithms are the algorithm of Myers and Miller (Myers and Miller 1988), the local homology algorithm of Smith et al. (Smith and Waterman 1981); the homology alignment algorithm of Needleman and Wunsch (Needleman and Wunsch 1970)); the search-for-similarity-method of Pearson and Lipman (Pearson and Lipman 1988).); the algorithm of Karlin and Altschul (Karlin and Altschul 1990), modified as in Karlin and Altschul (Karlin and Altschul 1993)). Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA (Pearson and Lipman 1988).); and TFASTA in the Wisconsin Genetics Software Package, Version 8 (available from Genetics Computer Group (GCG), 575 Science Drive, Madison, Wis., USA). Alignments using these programs can be performed using the default parameters and allowing a user to identify database sequences that resemble the reference sequence (query sequence) above a certain threshold of confidence.

Algorithms and programs for comparing primary biological sequence information between any two sequences typically provide an output comprising percent identity between the sequence retrieved and the reference sequence.

A person skilled in the art would understand that identity between sequences is typically measured by a process that comprises the steps of aligning the two polypeptide or polynucleotide sequences to form aligned sequences, then detecting the number of matched characters, i.e. characters similar or identical between the two aligned sequences, and calculating the total number of matched characters divided by the total number of aligned characters in each polypeptide or polynucleotide sequence, including gaps. The similarity result is expressed as a percentage of identity.

As used herein, “percentage of sequence identity” means the value determined by comparing two optimally aligned sequences over a comparison window, wherein the portion of the polynucleotide sequence in the comparison window may comprise additions or deletions (gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “genetic modification” means any process that adds, deletes, alters, or disrupts an endogenous nucleotide sequence and includes, but is not limited to viral mediated gene transfer, liposome mediated transfer, transformation, transfection and transduction, e.g., viral mediated gene transfer such as the use of vectors based on DNA viruses such as lentivirus, adenovirus, retroviruses, adeno-associated virus and herpes virus. Also included are methods of engineering using, for example CRISPR/CAS9 systems for modification.

“Variant” refers to polypeptides having amino acid sequences that differ to some extent from a native sequence polypeptide. Ordinarily, amino acid sequence variants will possess at least about 80% sequence identity, more preferably, at least about 90% homologous by sequence. The amino acid sequence variants may possess substitutions, deletions, and/or insertions at certain positions within the reference amino acid sequence.

“Introducing,” “introduce,” “introduced” (and grammatical variations thereof) in the context of a polynucleotide and/or polypeptide of interest means presenting a nucleotide sequence of interest (e.g., polynucleotide, a nucleic acid construct, and/or a guide nucleic acid) and/or polypeptide of interest to a host organism or cell of said organism (e.g., a mammalian cell) in such a manner that the nucleotide sequence and/or polypeptide gains access to the interior of a cell.

A “heterologous” or a “recombinant” nucleotide sequence is a nucleotide sequence not naturally associated with a host cell into which it is introduced, including non-naturally occurring multiple copies of a naturally occurring nucleotide sequence. For example, a heterologous polynucleotide encoding a Rac protein or portion thereof can be a nucleic acid sequence that is not naturally present in a phagocytic cell in which it is present and/or can be an additional nucleic acid sequence compared to the presence of a naturally occurring nucleotide sequence in a phagocytic cell in which it is present.

A “homologous” nucleic acid nucleotide sequence, polypeptide or amino acid sequence is a nucleotide sequence naturally associated with a host cell into which it is introduced. A homologous nucleic acid comprises a “native” nucleic acid, nucleotide sequence, polypeptide or amino acid sequence, which refer to a naturally occurring or endogenous nucleic acid, nucleotide sequence, polypeptide or amino acid sequence as will be understood by a skilled person.

The term “antibody” encompasses monoclonal antibodies, polyclonal antibodies, as well as hybrid antibodies, altered antibodies, chimeric antibodies, and humanized antibodies. The term antibody includes: hybrid (chimeric) antibody molecules (see, for example, Winter et al. (1991) Nature 349:293-299; and U.S. Pat. No. 4,816,567); bispecific antibodies, bispecific T cell engager antibodies (BiTE), trispecific antibodies, and other multispecific antibodies (see, e.g., Fan et al. (2015) J. Hematol. Oncol. 8:130, Krishnamurthy et al. (2018) Pharmacol Ther. 185:122-134), F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (scFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

The term “antigen-binding fragment” as used herein refers to any antibody fragment that specifically binds to a target antigen including, but not limited to, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (bivalent diabody), a multispecific antibody formed from a portion of an antibody including one or more complementarity determining regions (CDRs).

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity. VL and VH sequences can be reformatted as fragments, as single chain binding domains, linked to chimeric antigen receptors, and the like.

The term “antigen binding domain” refers to a domain that specifically binds to a target antigen. The antigen binding domain region of an antibody comprises a heavy-chain variable domain (VH) and a light-chain variable domain (VL) in non-covalent association as a single polypeptide or as a dimer. The three complementarity-determining regions of the heavy chain variable domain (CDR H1, H2, H3) and three complementarity-determining regions of the light chain variable domain (CDR L1, L2, L3) interact to define an antigen-binding site on the surface of an antibody. Collectively, the six CDRs of the light chain and heavy chain variable domains confer antigen-binding specificity to an antibody. An antigen binding domain region of a CAR may comprise all six CDRs of an antibody or a single variable domain or half of an Fv fragment comprising only three CDRs specific for an antigen, which still retains the ability to recognize and bind the target antigen. In some embodiments, the antigen-binding domain binds to one or more target antigens expressed on the surface of a target cell (e.g., cell surface markers). Exemplary lymphoma cell surface markers that can act as an antigen that binds to the antigen binding domain of a CAR include, without limitation, CD2, CD3, CD4, CD5, CD10, CD15, CD19, CD20, CD22, CD30, CD43, CD79a, BCL-2, BCL6, Pax-5, TdT, LCA, Oct-2, BOB.1, Ki67, and Epstein-Barr virus-latent membrane protein (EBV-LMP).

Chimeric Antigen Receptor (CAR). The CAR architecture may be any suitable architecture, as known in the art, comprising a transmembrane domain linked to an extracellular antigen-binding domain and an intracellular engulfment signaling domain, wherein the transmembrane domain is positioned between the extracellular antigen-binding domain and the intracellular engulfment signaling domain. See, e.g., International Patent Application Publication No. WO 2021/217087, U.S. Patent Application Publication No. 2020/0239592, U.S. Patent Application Publication No. 2020/0055917, U.S. Pat. No. 10,125,193, and Morrissey et al. (2018) Elife 7:e36688; herein incorporated by reference in their entireties.

The antigen-binding domain includes any naturally occurring, synthetic, semi-synthetic, or recombinantly produced binding partner for a target lymphoma marker or antigen of interest. In some embodiments, the binding region is an antigen-binding region, such as an antibody or functional binding domain or antigen-binding fragment thereof. The antigen-binding region of the CAR can include any domain that binds to the antigen and may include, but is not limited to, a monoclonal antibody, a polyclonal antibody, a synthetic antibody, a human antibody, a humanized antibody, a non-human antibody, a single-chain antibody, and any antigen-binding fragment thereof. Thus, in some embodiments, the antigen binding domain portion includes a mammalian antibody or an antigen-binding fragment thereof. An antigen-binding domain may comprise an antigen-binding fragment (Fab), a single-chain variable fragment (scFv), a nanobody, a VH domain, a VL domain, a single domain antibody (sdAb), a shark variable domain of a new antigen receptor (VNAR), a single variable domain on a heavy chain (VHH), a bispecific antibody, or a diabody; or a functional antigen-binding fragment thereof. In some embodiments, the antigen-binding domain is derived from the same cell type or the same species in which the CAR will ultimately be used. For example, for use in humans, the antigen-binding domain of the CAR may include a human antibody, a humanized antibody, or an antigen-binding fragment thereof.

In some embodiments, the antigen binding domain is derived from a single chain antibody that selectively binds to a target antigen. In some embodiments, the antigen binding domain is provided by a single chain variable fragment (scFv). A scFv is a recombinant molecule in which the variable regions of the light and heavy immunoglobulin chains are connected in a single fusion polypeptide. Generally, the VH and VL sequences are joined by a linker sequence. See, for example, Ahmad (2012) Clinical and Developmental Immunology Article ID 980250, herein specifically incorporated by reference. In principle, there are no particular limitations to the length and/or amino acid composition of the linker peptide joining the VH and VL sequences. In some embodiments, any arbitrary single-chain peptide including about 1 to 100 amino acid residues (e.g., 2, 3, 4, 5, 6, 7, 8, 9, 10, 11, 12, 13, 14, 15, 16, 17, 18, 19, 20, etc. amino acid residues) can be used as a peptide linker. In some embodiments, the linker peptide sequence includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the linker peptide sequence includes about 1 to 10, about 5 to 15, about 10 to 20, about 15 to 25, about 20 to 40, about 30 to 50, about 40 to 60, about 50 to 70 amino acid residues. In some embodiments, the linker peptide sequence includes about 40 to 70, about 50 to 80, about 60 to 80, about 70 to 90, or about 80 to 100 amino acid residues. In some embodiments, the linker peptide sequence includes about 1 to 10, about 5 to 15, about 10 to 20, about 15 to 25 amino acid residues

The transmembrane domain may be derived either from a natural or a synthetic source. Where the source is natural, the domain may be derived from any membrane-bound or transmembrane protein. In some embodiments, the transmembrane domain comprises at least the stalk and/or transmembrane region(s) of CD8, Megf10, FcRγ, Bai1, MerTK, TIM4, Stabilin-1, Stabilin-2, RAGE, CD300f, integrin subunit αv, Integrin subunit β5, CD36, LRP1, SCARF1, C1Qa, Axl, CD45, and/or CD86. In some embodiments, the CAR transmembrane domain is derived from a type I membrane protein, such as, but not limited to, CD3ζ, CD4, CD8, or CD28. In other embodiments, the transmembrane domain is synthetic, in which case it will include predominantly hydrophobic residues such as leucine, isoleucine, valine, phenylalanine, tryptophan, and alanine. In some embodiments, a triplet of phenylalanine, tryptophan and valine will be inserted at each end of a synthetic transmembrane domain.

In some embodiments, the CAR further comprises one or more linkers/spacers. For example, an extracellular spacer region may link the antigen binding domain to the transmembrane domain and/or an intracellular spacer region may link the intracellular engulfment signaling domain to the transmembrane domain. Various types of linkers may be used in the CARs described herein. In some embodiments, the linker includes a peptide linker/spacer sequence. In some embodiments, the length and amino acid composition of the linker peptide sequence is optimized to vary the orientation and/or proximity of the polypeptide domains to one another to optimize binding of the CAR to a target antigen and/or targeted phagocytic activity of an engineered phagocyte comprising the CAR. In some embodiments, the orientation and/or proximity of the polypeptide domains to one another can be varied as a “tuning” tool to achieve a tuning effect that would enhance or reduce the biological activity of the CAR.

In principle, there are no particular limitations to the length and/or amino acid composition of a linker peptide sequence. In some embodiments, a linker peptide sequence comprises about 1 to 100 amino acid residues, including any number of residues within this range such as 1, 2, 3, 4, 5, 6, 7, 8, 9, 10, 11, 12, 13, 14, 15, 16, 17, 18, 19, 20, 30, 40, 50, 60, 70, 80, 90, or 100 amino acid residues. In some embodiments, the linker peptide sequence includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the linker peptide sequence includes about 1 to 10, about 5 to 15, about 10 to 20, about 15 to 25, about 20 to 40, about 30 to 50, about 40 to 60, about 50 to 70 amino acid residues. In some embodiments, the linker peptide sequence includes about 40 to 70, about 50 to 80, about 60 to 80, about 70 to 90, or about 80 to 100 amino acid residues. In some embodiments, the linker peptide sequence includes about 1 to 10, about 5 to 15, about 10 to 20, about 15 to 25 amino acid residues. In some embodiments, the linker peptide sequence may include up to 300 amino acids, preferably 10 to 100 amino acids and most preferably 25 to 50 amino acids. In some embodiments, a short oligo- or polypeptide linker, preferably between 2 and 10 amino acids in length may form the linkage between the transmembrane domain and the intracellular engulfment signaling domain or extracellular antigen binding domain of the CAR.

In certain embodiments, the linker contains only glycine and/or serine residues (e.g., glycine-serine linker). Examples of such peptide linkers include: Gly, Ser; Gly Ser; Gly Gly Ser; Ser Gly Gly; Gly Gly Gly Ser (SEQ ID NO:2); Ser Gly Gly Gly; Gly Gly Gly Gly Ser (SEQ ID NO:3); Ser Gly Gly Gly Gly; Gly Gly Gly Gly Gly Ser (SEQ ID NO:4); Ser Gly Gly Gly Gly Gly; Gly Gly Gly Gly Gly Gly Ser (SEQ ID NO:5); Ser Gly Gly Gly Gly Gly Gly (SEQ ID NO:6); (Gly Gly Gly Gly Ser (SEQ ID NO:7))ₙ, wherein n is an integer of one or more; and (Ser Gly Gly Gly Gly (SEQ ID NO:8))ₙ, wherein n is an integer of one or more. In some embodiments, the linker peptides are modified such that the amino acid sequence GSG (that occurs at the junction of traditional Gly/Ser linker peptide repeats) is not present. For example, in some embodiments, the peptide linker includes an amino acid sequence selected from the group consisting of: (GGGXX)ₙGGGGS (SEQ ID NO:9) and GGGGS(XGGGS)ₙ (SEQ ID NO:10), where X is any amino acid that can be inserted into the sequence and not result in a polypeptide including the sequence GSG, and n is 0 to 4. In some embodiments, the sequence of a linker peptide is (GGGPS)ₙGGGGS (SEQ ID NO:11) and n is 0 to 4. In some other embodiments, the sequence of a linker peptide is (GGGGQ)ₙGGGGS (SEQ ID NO:12) and n is 0 to 4. In some other embodiments, the sequence of a linker peptide is (GGGGA)ₙGGGGS (SEQ ID NO:13) and n is 0 to 4. In yet some other embodiments, the sequence of a linker peptide is GGGGS(PGGGS)ₙ (SEQ ID NO:14), and n is 0 to 4. In some embodiments, a linker peptide of the disclosure comprises or consists of the amino acid sequence (GGGGA)₂GGGGS (SEQ ID NO15). In some embodiments, a linker peptide comprises or consists of the amino acid sequence (GGGGQ)₂GGGGS (SEQ ID NO16). In another embodiment, a linker peptide comprises or consists of the amino acid sequence (GGGPS)₂GGGGS (SEQ ID NO:17). In another embodiment, a linker peptide comprises or consists of the amino acid sequence GGGGS(PGGGS)₂ (SEQ ID NO:18). In yet a further embodiment, a linker peptide comprises or consists of the amino acid sequence GSGGS (SEQ ID NO:19) or SGGSGS (SEQ ID NO:20).

An “engulfment signaling domain” refers to an intracellular effector domain, which, upon binding of the target molecule (e.g., lymphoma antigen) targeted by the extracellular antigen-binding domain of a CAR expressed by a host cell (engineered phagocyte), activates one or more signaling pathways in the host cell resulting in engulfment, including, in specific embodiments, cytoskeletal rearrangement of the host cell and internalization of the target cell, microbe, or particle associated with the marker or antigen. In certain embodiments, an engulfment signaling domain activates one or more signaling pathways resulting in phagocytosis of the target cell (e.g., lymphoma cell), microbe, or particle. In certain embodiments, the engulfment signaling domain includes a primary engulfment signaling domain. In certain other embodiments, the engulfment signaling domain includes a primary engulfment signaling domain and a secondary engulfment signaling domain. A primary engulfment may be a homeostatic engulfment signaling domain or a pro-inflammatory engulfment signaling domain. In embodiments where the engulfment signaling domain includes a primary engulfment signaling domain and a secondary engulfment signaling domain, the primary engulfment signaling domain can be a homeostatic engulfment signaling domain or a pro-inflammatory engulfment signaling domain. Similarly, the secondary engulfment signaling domain can be selected from a homeostatic engulfment signaling domain or a pro-inflammatory engulfment signaling domain. In certain embodiments, the CAR includes a primary engulfment signaling domain and a secondary engulfment signaling domain that are both homeostatic engulfment signaling domains. In certain other embodiments, the CAR includes a primary engulfment signaling domain and a secondary engulfment signaling domain that are both pro-inflammatory engulfment signaling domains. In still other embodiments, the CAR includes a primary engulfment signaling domain that is a homeostatic engulfment signaling domain and a secondary engulfment signaling domain that is a pro-inflammatory engulfment signaling domain. In still other embodiments, the CAR includes a primary engulfment signaling domain that is a pro-inflammatory engulfment signaling domain and a secondary engulfment signaling domain that is a homeostatic engulfment signaling domain.

In some embodiments of the disclosure, the intracellular engulfment signaling domain includes at least 1, at least 2, at least 3, at least 4, or at least 5 immunoreceptor tyrosine-based activation motif (ITAM). Generally, any intracellular signaling domain including an ITAM can be suitably used for the construction of the CAR as described herein. An “ITAM,” as used herein, is a conserved protein motif that is generally present in the tail portion of signaling molecules expressed in many immune cells. The motif may include two repeats of the amino acid sequence YxxL/I separated by 6-8 amino acids, wherein each X is independently any amino acid, producing the conserved motif YxxL/Ix(6-8)YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. In some embodiments, the intracellular engulfment signaling domain comprises at least 1, at least 2, at least 3, at least 4, or at least 5 ITAMs independently selected from the ITAMs derived from CD3ζ, FcRγ, Megf10, FcRγ, and combinations thereof.

In some embodiments, the intracellular engulfment signaling domain is capable of mediating an endogenous phagocytic signaling pathway. In some embodiments, the intracellular domain of the CAR includes a domain responsible for signal activation and/or transduction. Non-limiting examples of an intracellular domain suitable for the CARs disclosed herein include, the cytoplasmic portion of a surface receptor, co-stimulatory molecule, and any molecule that acts in concert to initiate signal transduction in the phagocytic cell (e.g., monocyte, macrophage or dendritic cell), as well as any derivative or variant of these elements and any synthetic sequence that has the same functional capability. In some embodiments, the CAR comprises at least one intracellular domain derived from an engulfment receptor such as, e.g., Megf10, FcRγ, Bai1, MerTK, TIM4, Stabilin-1, Stabilin-2, RAGE, CD300f, Integrin subunit αv, Integrin subunit β5, CD36, LRP1, SCARF1, C1Qa, and/or Axl.

The term “homeostatic engulfment signaling domain” refers to an effector domain that (i) stimulates engulfment of a targeted cell, microbe, or particle and (ii) is derived from an endogenous receptor or signaling molecule that typically stimulates an inflammatory or immunogenic response. In some embodiments, a homeostatic engulfment signaling domain stimulates host cell secretion of anti-inflammatory and/or immunosuppressive cytokines, such as, for example, TGF-β and IL-10. In certain embodiments, stimulation of homeostatic engulfment signaling dampens, attenuates, or resolves inflammation in the local tissue milieu. A homeostatic engulfment signaling domain can also be referred to as a “non-inflammatory” engulfment signaling domain or a “non-immunogenic” engulfment signaling domain.

A “pro-inflammatory engulfment signaling domain” refers to an effector domain that (i) stimulates engulfment of a targeted cell, microbe, or particle and (ii) is derived from an endogenous receptor or signaling molecule that typically stimulates one or more of (a) host cell secretion of inflammatory cytokines, such as, for example, TNFα, IL-1, IL-6, IL-12, and IL-23, (b) host cell secretion of inflammatory chemokines, such as, for example, CCL5 (RANTES), CXCL9, and CXCL10, (c) upregulation of cell surface co-stimulatory markers, such as, for example, CD80, CD86, HLA-DR, CD40, HVEM, and 4-1BBL, and (d) activation of one or more signaling cascades, such as NF-κB, that induce, potentiate, or complement chemotherapies, antibody-based immune therapies, or cellular therapies, such as, for example, T cell targeted therapies. In certain embodiments, stimulation of pro-inflammatory engulfment signaling promotes inflammation in the local tissue milieu. A pro-inflammatory engulfment signaling domain can also be referred to as an “immunogenic” engulfment signaling domain or an “inflammatory” engulfment signaling domain. In some embodiments, the CAR includes an amino acid sequence for one or more signal peptides. Generally, there are no specific limitations with respect to the position where the signal peptide is operably linked, e.g., fused, to the CAR polypeptide. In some embodiments, the signal peptide is operably linked upstream (e.g., N-terminally) of the extracellular antigen-binding domain. The signal peptide can generally be any signal peptide known in the art. Non-limiting examples of signal peptides suitable for the compositions and methods disclosed herein include signal peptides derived from CD8, Megf10, FcRγ, Bai1, MerTK, TIM4, Stabilin-1, Stabilin-2, RAGE, CD300f, Integrin subunit αv, Integrin subunit β5, CD36, LRP1, SCARF1, C1Qa, and Axl.

The term “operably linked”, as used herein, denotes a physical or functional linkage between two or more elements, e.g., polypeptide sequences or polynucleotide sequences, which permits them to operate in their intended fashion. For example, an operable linkage between a polynucleotide of interest and a regulatory sequence (for example, a promoter) is a functional link that allows for expression of the polynucleotide of interest. In this sense, the term “operably linked” refers to the positioning of a regulatory region and a coding sequence to be transcribed so that the regulatory region is effective for regulating transcription or translation of the coding sequence of interest. In some embodiments disclosed herein, the term “operably linked” denotes a configuration in which a regulatory sequence is placed at an appropriate position relative to a sequence that encodes a polypeptide or functional RNA such that the control sequence directs or regulates the expression or cellular localization of the mRNA encoding the polypeptide, the polypeptide, and/or the functional RNA. Thus, a promoter is in operable linkage with a nucleic acid sequence if it can mediate transcription of the nucleic acid sequence. Operably linked elements may be contiguous or non-contiguous. In addition, in the context of a polypeptide, “operably linked” refers to a physical linkage (e.g., directly or indirectly linked) between amino acid sequences (e.g., different segments, regions, or domains) to provide for a described activity of the polypeptide. In the present disclosure, various segments, regions, or domains may be operably linked to retain proper folding, processing, targeting, expression, binding, and other functional properties of a polypeptide in a cell. Unless stated otherwise, various regions, domains, and segments of a CAR of the disclosure are operably linked to each other. Operably linked regions, domains, and segments of the CAR of the disclosure may be contiguous or non-contiguous (e.g., linked to one another through a linker).

The term “phagocyte promoter” refers to a nucleotide sequence that drives or regulates expression in phagocytes. Promoters specific to mononuclear phagocyte system (MPS) including macrophages, neutrophils, dendritic cells, and osteoclasts will constitute phagocyte promoters. Examples of such promoters include, but are not limited to, the CSF-1, CD68, CD11c, DC-SIGN, DC-STAMP, Langerin, and human neutrophil elastase promoters, and any synthetic promoter containing elements of a phagocyte system designed to achieve high level of expression in phagocytic cells.

In some embodiments, activated phagocytes in accordance of the present disclosure are phagocytes genetically engineered to comprise an activated RAC (e.g., RAC2) gene in a configuration allowing constitutive or conditional expression in a genetically engineered phagocyte.

The term “constitutive promoter” refers to an unregulated promoter that allows for continual transcription of its associated genes. Exemplary mammalian constitutive promoters that can be used for expression in mammalian cells include CMV from human cytomegalovirus, EF1a from human elongation factor 1 alpha, SV40 from the simian vacuolating virus 40, PGK1 from phosphoglycerate kinase gene, Ubc from human ubiquitin C gene, human beta actin, CAAG, Synl and others identifiable to those skilled in the art.

The term “conditional promoter” refers to a promoter with activity regulatable or controlled by endogenous transcription factors or exogenous inputs such as chemical or thermal inducers or optical induction. Examples of mammalian conditional promoters include inducible promoters based on exogenous agents such as TET (tetracycline-response elements, TET-ON/TET-OFF), Lac, dCas-transactivator, Zinc-finger-TF, TALENs-ZF Gal4-uas, synNotch and inducible promoters based on endogenous signals TNF-alpha, cFOS and others identifiable to a skilled person.

The term “regulatory sequence” or “regulatory regions” as described herein indicate a segment of a nucleic acid molecule which is capable of increasing or decreasing transcription or translation of a gene within an organism either in vitro or in vivo. In particular, coding regions of the activated Rac genes herein described comprise one or more protein coding regions which when transcribed and translated produce a polypeptide. Regulatory regions of a gene herein described comprise promoters, transcription factor binding sites, binding sites operators, activator binding sites, protein-protein binding domains, RNA binding domains, DNA binding domains, repressors, enhancers, insulators, silencers and additional regulatory regions that can alter gene expression in response to developmental and/or external stimuli as will be recognized by a person skilled in the art.

Regulatory regions controlling expression of a gene in a phagocyte are herein indicated as “phagocyte regulatory regions”.

In some embodiments, the configuration of a genetically engineered phagocyte can include constitutive, conditional and/or phagocyte promoters (examples include CSF-1, CD68, p47phox promoter etc.) containing regulatory sequences for cell type specific expression (e.g., macrophage-specific transcription factor PU1, Ets family transcription factors and STAT1, C/EBP-α, C/EBP-δ, IRF9, KLF6, and NF-κB transcription factors), DNA and RNA-binding proteins (e.g. EWS and FUS/TLS) upstream of naturally occurring, overexpressed or activated Rac genes. The stoichiometric configuration of various genetic elements in the engineered phagocyte can be optimized by introducing a reporter (such as GFP) along with the other genetic elements and assessing the expression of the reporter in different stoichiometric configurations such as by introducing multiple copies of promoters, enhancers, transcription factor binding sites and additional elements identifiable by a skilled person.

As used herein, a “vector” may be any agent capable of delivering or maintaining nucleic acid in a host cell, and includes viral vectors (e.g. retroviral vectors, lentiviral vectors, adenoviral vectors, or adeno-associated viral vectors), plasmids, naked nucleic acids, nucleic acids complexed with polypeptide or other molecules and nucleic acids immobilized onto solid phase particles. The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art. Transcription of the DNA encoding the polypeptides of the present invention by higher eukaryotes is increased by inserting an enhancer sequence into the vector. Enhancers are cis-acting elements of DNA, usually about from 10 to 300 bp that act on a promoter to increase its transcription. Examples including the SV40 enhancer on the late side of the replication origin, a cytomegalovirus early promoter enhancer, the polyoma enhancer on the late side of the replication origin, and adenovirus enhancers.

An “expression cassette” comprises a DNA coding sequence (e.g., a nucleotide sequence encoding a CAR or Rac GTPase) operably linked to a transcriptional control element. “Operably linked” refers to a juxtaposition wherein the components so described are in a relationship permitting them to function in their intended manner. For instance, a control element is operably linked to a coding sequence (and likewise the coding sequence is operably linked to the control element) if the control element affects transcription/expression of the coding sequence. As would be readily understood by one of ordinary skill in the art, a nucleotide sequence can be operably linked to more than one control element (e.g., a promoter and an enhancer).

The terms “recombinant expression vector,” “expression vector” and similar terms of the art are used interchangeably herein to refer to a DNA molecule comprising a vector and at least one insert, where the insert includes an expression cassette (e.g., expressing a CAR or Rac GTPase). Recombinant expression vectors can be generated for the purpose of expressing and/or propagating the insert(s) (e.g., in bacteria or mammalian cells), or for the construction of other recombinant nucleotide sequences. In some cases, a subject nucleic acid (e.g., an expression cassette, an expression vector, a viral expression vector, a linear expression vector, a circular expression vector, a plasmid, and the like) includes a promoter operably linked to a nucleotide sequence encoding a CAR or Rac GTPase.

Suitable expression vectors include, but are not limited to, viral vectors (e.g., viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (AAV) (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (HIV) (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); lentiviral vector (e.g., a CD511B-1 lentiviral expression vector modified to include a promoter operably linked to a nucleotide sequence encoding a CAR or Rac GTPase); and the like.

In some embodiments, the activated Rac, the phagocyte promoter and the additional phagocyte regulatory regions can be comprised as a part of a gene expression cassette.

The term “gene cassette” as used herein indicated a mobile genetic element that contains at least one gene and a recombination site. Accordingly, a gene cassette can contain a single gene or multiple genes possibly organized in an operon structure. A gene cassette can be transferred from one DNA sequence (usually on a vector) to another by ‘cutting’ the fragment out using restriction enzymes or transposase, cripr, viral and/or recombinase enzymes and other nucleases and ‘pasting’ it back into the new context or other molecular biology and cloning techniques (e.g. pcr, CRISPR, TALENs, ZFN). Gene cassettes can move around within an organism's genome or be transferred to another organism in the environment via horizontal gene transfer.

A “gene expression cassette” is a gene cassette comprising regulatory sequence to be expressed by a transfected cell. Following transformation, the expression cassette directs the cell's machinery to make RNA and proteins. Some expression cassettes are designed for modular cloning of protein-encoding sequences so that the same cassette can be altered to make different proteins. An expression cassette is composed of one or more genes and the sequences controlling their expression. An expression cassette typically comprises at least three components: a promoter sequence, an open reading frame, and a 3′ untranslated region that, in eukaryotes, usually contains a polyadenylation site. An expression cassette can be formed by manipulable fragment of DNA carrying and capable of expressing, one or more genes of interest optionally located between one or more sets of restriction sites. Gene expression cassettes as used herein typically comprise further regulatory sequences additional to the prompter to regulate the expression of the gene or genes within the open reading frame herein also indicated as coding region of the cassette.

“Gene transfer” or “gene delivery” refers to methods or systems for reliably inserting DNA or RNA of interest into a host cell. Such methods can result in transient expression of non-integrated transferred DNA, mRNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, adenoviruses, lentiviruses, alphaviruses, pox viruses, and vaccinia viruses.

The terms “transformation” or “transfection” may be used interchangeably and as used herein refer to the introduction of a nucleic acid into a cell. Transformation of a cell may be stable or transient. Thus, in some embodiments, a host cell or host organism may be stably transformed with a polynucleotide/nucleic acid molecule of the invention. In some embodiments, a host cell or host organism may be transiently transformed with a nucleic acid construct of the invention.

“Transient transformation” in the context of a polynucleotide means that a polynucleotide is introduced into the cell and does not integrate into the genome of the cell.

By “stably introducing” or “stably introduced” in the context of a polynucleotide introduced into a cell is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and the plastid genome, and therefore includes integration of the nucleic acid into, for example, the chloroplast or mitochondrial genome. Stable transformation as used herein can also refer to a transgene that is maintained extrachromosomally, for example, as a minichromosome or a plasmid.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a mammal). Stable transformation of a cell can be detected by, for example, a Northern blot hybridization assay of RNA of the cell with nucleic acid sequences which specifically hybridize with a nucleotide sequence of a transgene introduced into a host organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods. Transformation can also be detected by direct sequencing and/or hybridization protocols well known in the art.

Accordingly, in some embodiments, nucleotide sequences, polynucleotides, nucleic acid constructs, and/or expression cassettes of the invention may be expressed transiently and/or they can be stably incorporated into the genome of the host organism.

In particular, in embodiments of activated Rac genes herein described, the gene expression cassettes can comprise one or more activated Rac genes under control of regulatory regions capable of operating in the phagocyte and are thus configured to provide an activated phagocyte.

### Engineered Phagocytes

Engineered phagocytes are provided for administration to an individual for treatment of lymphoma. As used herein, the term “engineered” is intended to refer to a phagocyte into which an exogenous nucleic acid sequence, such as, for example, a vector or mRNA, has been introduced. Therefore, engineered phagocytes are distinguishable from naturally occurring cells which do not contain a recombinantly introduced nucleic acid. The phagocytes are engineered to express a chimeric antigen receptor (CAR) that specifically binds to an antigen present on lymphoma cells. The CAR localizes the engineered phagocytes to sites where lymphoma cells are present. In some embodiments, phagocytic activity of the phagocyte is enhanced by further engineering the phagocyte to express a hyperactive Rac GTPase. The innovative combination of a CAR with a hyperactive Rac significantly enhances phagocytic activity and is generalizable to the treatment of many additional diseases, including bacterial infections (e.g., multidrug resistant bacteria), viral infections, and autoimmune diseases, and the removal of unwanted cells or substances from the body.

The CAR architecture may be any suitable architecture, as known in the art, comprising a transmembrane domain linked to an extracellular antigen binding domain and an intracellular engulfment signaling domain, wherein the transmembrane domain is positioned between the extracellular antigen binding domain and the intracellular engulfment signaling domain. See, e.g., International Patent Application Publication No. WO 2021/217087, U.S. Patent Application Publication No. 2020/0239592, U.S. Patent Application Publication No. 2020/0055917, U.S. Pat. No. 10,125,193, and Morrissey et al. (2018) Elife 7:e36688; herein incorporated by reference in their entireties.

The antigen binding domain includes any naturally occurring, synthetic, semi-synthetic, or recombinantly produced binding partner for a target lymphoma marker or antigen of interest. In some embodiments, the binding region is an antigen-binding region, such as an antibody or functional binding domain or antigen-binding fragment thereof. The antigen-binding region of the CAR can include any domain that binds to the antigen and may include, but is not limited to, a monoclonal antibody, a polyclonal antibody, a synthetic antibody, a human antibody, a humanized antibody, a non-human antibody, a single-chain antibody, and any antigen-binding fragment thereof. Thus, in some embodiments, the antigen binding domain portion includes a mammalian antibody or an antigen-binding fragment thereof. An antigen-binding domain may comprise an antigen-binding fragment (Fab), a single-chain variable fragment (scFv), a nanobody, a VH domain, a VL domain, a single domain antibody (sdAb), a shark variable domain of a new antigen receptor (VNAR), a single variable domain on a heavy chain (VHH), a bispecific antibody, or a diabody; or a functional antigen-binding fragment thereof. In some embodiments, the antigen-binding domain is derived from the same cell type or the same species in which the CAR will ultimately be used. For example, for use in humans, the antigen-binding domain of the CAR may include a human antibody, a humanized antibody, or an antigen-binding fragment thereof.

In some embodiments, the antigen binding domain is derived from a single chain antibody that selectively binds to a target antigen. In some embodiments, the antigen binding domain is provided by a single chain variable fragment (scFv). A scFv is a recombinant molecule in which the variable regions of the light and heavy immunoglobulin chains are connected in a single fusion polypeptide. Generally, the VH and VL sequences are joined by a linker sequence. See, for example, Ahmad (2012) Clinical and Developmental Immunology Article ID 980250, herein specifically incorporated by reference. In principle, there are no particular limitations to the length and/or amino acid composition of the linker peptide joining the VH and VL sequences. In some embodiments, any arbitrary single-chain peptide including about 1 to 100 amino acid residues (e.g., 2, 3, 4, 5, 6, 7, 8, 9, 10, 11, 12, 13, 14, 15, 16, 17, 18, 19, 20, etc. amino acid residues) can be used as a peptide linker. In some embodiments, the linker peptide sequence includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the linker peptide sequence includes about 1 to 10, about 5 to 15, about 10 to 20, about 15 to 25, about 20 to 40, about 30 to 50, about 40 to 60, about 50 to 70 amino acid residues. In some embodiments, the linker peptide sequence includes about 40 to 70, about 50 to 80, about 60 to 80, about 70 to 90, or about 80 to 100 amino acid residues. In some embodiments, the linker peptide sequence includes about 1 to 10, about 5 to 15, about 10 to 20, about 15 to 25 amino acid residues.

The transmembrane domain connects the extracellular antigen binding domain to the intracellular domain. In some embodiments, the transmembrane domain is operably linked downstream (e.g., C-terminally) of the extracellular domain and upstream of the at least one copy of the intracellular engulfment signaling domain. The transmembrane domain may be derived either from a natural or from a synthetic source. Where the source is natural, the domain may be derived from any membrane-bound or transmembrane protein. In some embodiments, the transmembrane domain comprises at least the stalk and/or transmembrane region(s) of CD8, Megf10, FcRγ, Bai1, MerTK, TIM4, Stabilin-1, Stabilin-2, RAGE, CD300f, integrin subunit αv, Integrin subunit β5, CD36, LRP1, SCARF1, C1Qa, Axl, CD45, and/or CD86. In some embodiments, the CAR transmembrane domain (TM) is derived from a type I membrane protein, such as, but not limited to, CD3ζ, CD4, CD8, or CD28. In other embodiments, the transmembrane domain is synthetic, in which case it will include predominantly hydrophobic residues such as leucine, isoleucine, valine, phenylalanine, tryptophan, and alanine. In some embodiments, a triplet of phenylalanine, tryptophan and valine will be inserted at each end of a synthetic transmembrane domain.

In some embodiments, the CAR further comprises one or more linkers/spacers. For example, an extracellular spacer region may link the antigen binding domain to the transmembrane domain and/or an intracellular spacer region may link the intracellular engulfment signaling domain to the transmembrane domain. Various types of linkers may be used in the CARs described herein. In some embodiments, the linker includes a peptide linker/spacer sequence. In some embodiments, the length and amino acid composition of the linker peptide sequence is optimized to vary the orientation and/or proximity of the polypeptide domains to one another to optimize binding of the CAR to a target antigen and/or targeted phagocytic activity of an engineered phagocyte comprising the CAR. In some embodiments, the orientation and/or proximity of the polypeptide domains to one another can be varied as a “tuning” tool to achieve a tuning effect that would enhance or reduce the biological activity of the CAR.

In principle, there are no particular limitations to the length and/or amino acid composition of a linker peptide sequence. In some embodiments, a linker peptide sequence comprises about 1 to 100 amino acid residues, including any number of residues within this range such as 1, 2, 3, 4, 5, 6, 7, 8, 9, 10, 11, 12, 13, 14, 15, 16, 17, 18, 19, 20, 30, 40, 50, 60, 70, 80, 90, or 100 amino acid residues. In some embodiments, the linker peptide sequence includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the linker peptide sequence includes about 1 to 10, about 5 to 15, about 10 to 20, about 15 to 25, about 20 to 40, about 30 to 50, about 40 to 60, about 50 to 70 amino acid residues. In some embodiments, the linker peptide sequence includes about 40 to 70, about 50 to 80, about 60 to 80, about 70 to 90, or about 80 to 100 amino acid residues. In some embodiments, the linker peptide sequence includes about 1 to 10, about 5 to 15, about 10 to 20, about 15 to 25 amino acid residues. In some embodiments, the linker peptide sequence may include up to 300 amino acids, preferably 10 to 100 amino acids and most preferably 25 to 50 amino acids. In some embodiments, a short oligo- or polypeptide linker, preferably between 2 and 10 amino acids in length may form the linkage between the transmembrane domain and the intracellular engulfment signaling domain or extracellular antigen binding domain of the CAR.

In certain embodiments, the linker contains only glycine and/or serine residues (e.g., glycine-serine linker). Examples of such peptide linkers include: Gly, Ser; Gly Ser; Gly Gly Ser; Ser Gly Gly; Gly Gly Gly Ser (SEQ ID NO:2); Ser Gly Gly Gly; Gly Gly Gly Gly Ser (SEQ ID NO:3); Ser Gly Gly Gly Gly; Gly Gly Gly Gly Gly Ser (SEQ ID NO:4); Ser Gly Gly Gly Gly Gly; Gly Gly Gly Gly Gly Gly Ser (SEQ ID NO:5); Ser Gly Gly Gly Gly Gly Gly (SEQ ID NO:6); (Gly Gly Gly Gly Ser (SEQ ID NO:7))ₙ, wherein n is an integer of one or more; and (Ser Gly Gly Gly Gly (SEQ ID NO:8))ₙ, wherein n is an integer of one or more. In some embodiments, the linker peptides are modified such that the amino acid sequence GSG (that occurs at the junction of traditional Gly/Ser linker peptide repeats) is not present. For example, in some embodiments, the peptide linker includes an amino acid sequence selected from the group consisting of: (GGGXX)ₙGGGGS (SEQ ID NO:9) and GGGGS(XGGGS)ₙ (SEQ ID NO:10), where X is any amino acid that can be inserted into the sequence and not result in a polypeptide including the sequence GSG, and n is 0 to 4. In some embodiments, the sequence of a linker peptide is (GGGPS)ₙGGGGS (SEQ ID NO:11) and n is 0 to 4. In some other embodiments, the sequence of a linker peptide is (GGGGQ)ₙGGGGS (SEQ ID NO:12) and n is 0 to 4. In some other embodiments, the sequence of a linker peptide is (GGGGA)ₙGGGGS (SEQ ID NO:13) and n is 0 to 4. In yet some other embodiments, the sequence of a linker peptide is GGGGS(PGGGS)ₙ (SEQ ID NO:14), and n is 0 to 4. In some embodiments, a linker peptide of the disclosure comprises or consists of the amino acid sequence (GGGGA)₂GGGGS (SEQ ID NO15). In some embodiments, a linker peptide comprises or consists of the amino acid sequence (GGGGQ)₂GGGGS (SEQ ID NO16). In another embodiment, a linker peptide comprises or consists of the amino acid sequence (GGGPS)₂GGGGS (SEQ ID NO:17). In another embodiment, a linker peptide comprises or consists of the amino acid sequence GGGGS(PGGGS)₂ (SEQ ID NO:18). In yet a further embodiment, a linker peptide comprises or consists of the amino acid sequence GSGGS (SEQ ID NO:19) or SGGSGS (SEQ ID NO:20).

An “engulfment signaling domain” refers to an intracellular effector domain, which, upon binding of the target molecule (e.g., lymphoma antigen) targeted by the extracellular domain of a CAR expressed by a host cell (engineered phagocyte), activates one or more signaling pathways in the host cell resulting in engulfment, including, in specific embodiments, cytoskeletal rearrangement of the host cell and internalization of the target cell, microbe, or particle associated with the marker or antigen. In certain embodiments, an engulfment signaling domain activates one or more signaling pathways resulting in phagocytosis of the target cell (e.g., lymphoma cell), microbe, or particle. In certain embodiments, the engulfment signaling domain includes a primary engulfment signaling domain. In certain other embodiments, the engulfment signaling domain includes a primary engulfment signaling domain and a secondary engulfment signaling domain. A primary engulfment may be a homeostatic engulfment signaling domain or a pro-inflammatory engulfment signaling domain. In embodiments where the engulfment signaling domain includes a primary engulfment signaling domain and a secondary engulfment signaling domain, the primary engulfment signaling domain can be a homeostatic engulfment signaling domain or a pro-inflammatory engulfment signaling domain. Similarly, the secondary engulfment signaling domain can be selected from a homeostatic engulfment signaling domain or a pro-inflammatory engulfment signaling domain. In certain embodiments, the CAR includes a primary engulfment signaling domain and a secondary engulfment signaling domain that are both homeostatic engulfment signaling domains. In certain other embodiments, the CAR includes a primary engulfment signaling domain and a secondary engulfment signaling domain that are both pro-inflammatory engulfment signaling domains. In still other embodiments, the CAR includes a primary engulfment signaling domain that is a homeostatic engulfment signaling domain and a secondary engulfment signaling domain that is a pro-inflammatory engulfment signaling domain. In still other embodiments, the CAR includes a primary engulfment signaling domain that is a pro-inflammatory engulfment signaling domain and a secondary engulfment signaling domain that is a homeostatic engulfment signaling domain.

In some embodiments of the disclosure, the intracellular engulfment signaling domain includes at least 1, at least 2, at least 3, at least 4, or at least 5 immunoreceptor tyrosine-based activation motif (ITAM). Generally, any intracellular signaling domain including an ITAM can be suitably used for the construction of the CAR as described herein. An “ITAM,” as used herein, is a conserved protein motif that is generally present in the tail portion of signaling molecules expressed in many immune cells. The motif may include two repeats of the amino acid sequence YxxL/I separated by 6-8 amino acids, wherein each X is independently any amino acid, producing the conserved motif YxxL/Ix(6-8)YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. In some embodiments, the intracellular engulfment signaling domain comprises at least 1, at least 2, at least 3, at least 4, or at least 5 ITAMs independently selected from the ITAMs derived from CD3ζ, FcRγ, Megf10, FcRγ, and combinations thereof.

In some embodiments, the intracellular engulfment signaling domain is capable of mediating an endogenous phagocytic signaling pathway. In some embodiments, the intracellular domain of the CAR includes a domain responsible for signal activation and/or transduction. Non-limiting examples of an intracellular domain suitable for the CARs disclosed herein include, the cytoplasmic portion of a surface receptor, co-stimulatory molecule, and any molecule that acts in concert to initiate signal transduction in the phagocytic cell (e.g., monocyte, macrophage or dendritic cell), as well as any derivative or variant of these elements and any synthetic sequence that has the same functional capability. In some embodiments, the CAR comprises at least one intracellular domain derived from an engulfment receptor such as, e.g., Megf10, FcRγ, Bai1, MerTK, TIM4, Stabilin-1, Stabilin-2, RAGE, CD300f, Integrin subunit αv, Integrin subunit β5, CD36, LRP1, SCARF1, C1Qa, and/or Axl.

The term “homeostatic engulfment signaling domain” refers to an effector domain that (i) stimulates engulfment of the targeted cell, microbe, or particle and (ii) is derived from an endogenous receptor or signaling molecule that typically stimulates an inflammatory or immunogenic response. In some embodiments, a homeostatic engulfment signaling domain stimulates host cell secretion of anti-inflammatory and/or immunosuppressive cytokines, such as, for example, TGF-β and IL-10. In certain embodiments, stimulation of homeostatic engulfment signaling dampens, attenuates, or resolves inflammation in the local tissue milieu. A homeostatic engulfment signaling domain can also be referred to as a “non-inflammatory” engulfment signaling domain or a “non-immunogenic” engulfment signaling domain.

A “pro-inflammatory engulfment signaling domain” refers to an effector domain that (i) stimulates engulfment of the targeted cell, microbe, or particle and (ii) is derived from an endogenous receptor or signaling molecule that typically stimulates one or more of (a) host cell secretion of inflammatory cytokines, such as, for example, TNFα, IL-1, IL-6, IL-12, and IL-23, (b) host cell secretion of inflammatory chemokines, such as, for example, CCL5 (RANTES), CXCL9, and CXCL10, (c) upregulation of cell surface co-stimulatory markers, such as, for example, CD80, CD86, HLA-DR, CD40, HVEM, and 4-1BBL, and (d) activation of one or more signaling cascades, such as NF-κB, that induce, potentiate, or complement chemotherapies, antibody-based immune therapies, or cellular therapies, such as, for example, T cell targeted therapies. In certain embodiments, stimulation of pro-inflammatory engulfment signaling promotes inflammation in the local tissue milieu. A pro-inflammatory engulfment signaling domain can also be referred to as an “immunogenic” engulfment signaling domain or an “inflammatory” engulfment signaling domain.

In some embodiments, the CAR includes an amino acid sequence for one or more signal peptides. Generally, there are no specific limitations with respect to the position where the signal peptide is operably linked, e.g. fused, to the CAR polypeptide. In some embodiments, the signal peptide is operably linked upstream (e.g., N-terminally) of the extracellular antigen-binding domain. The signal peptide can generally be any signal peptide known in the art. Non-limiting examples of signal peptides suitable for the compositions and methods disclosed herein include signal peptides derived from CD8, Megf10, FcRγ, Bai1, MerTK, TIM4, Stabilin-1, Stabilin-2, RAGE, CD300f, Integrin subunit αv, Integrin subunit β5, CD36, LRP1, SCARF1, C1Qa, and Axl.

In some embodiments, the phagocyte is further engineered to express a hyperactive Rac GTPase. The term “hyperactive or “activated” as used herein with reference to a Rac protein refers to a Rac protein having a modification to its sequence resulting in increased Rac biological activity. One or more Rac biological activities may be enhanced by a mutation, including GTP binding, GTP hydrolysis and/or association with downstream effectors, which mediate Rac effects on various biological events, such as structural changes to the actin, cytoskeletal reorganization, cell growth, cell movement, translocation of glucose transforming vesicles, glucose uptake, antimicrobial cytotoxicity, the activation of protein kinases and additional events identifiable by a skilled person. Exemplary downstream effectors include, without limitation, the serine/threonine-protein kinase, Akt, also known as protein kinase B, as well as serine/threonine protein kinase, p65^{PAK}, also known as PAK1, and additional downstream effectors identifiable by a skilled person.

A hyperactive Rac protein may have one or more mutations that enhance one or more biological activities of a Rac protein. In some embodiments, the hyperactive Rac protein has a mutation (e.g., a point mutation) at one or more amino acid residues corresponding to amino acid residue number 11, 12, 28, 29, 30, 34, 62, 63, 92, and/or 157, wherein numbering of amino acid positions is relative to the reference sequence of SEQ ID NO 1. In some embodiments, a hyperactive Rac comprises one or more activating mutations selected from D11A, G12V/R, F28L, P29S, P29L, P29Q, PG(29,30)VD, P34H, E62K, D63V N92S, N92T N921, C157Y, wherein numbering of amino acid positions is relative to the reference sequence of SEQ ID NO 1. For a description of activating mutations that increase activity of Rac proteins, see, e.g., International Patent Application No. WO 2021217087 A1, herein incorporated by reference in its entirety.

In some embodiments, the phagocyte is engineered to express a hyperactive Rac 2 GTPase. RAC2 nucleic acid and protein sequences may be derived from any source. A number of RAC2 nucleic acid and protein sequences are known. A representative human RAC2 amino acid sequence is presented in SEQ ID NO:1, and additional representative sequences are listed in the National Center for Biotechnology Information (NCBI) database. See, for example, NCBI entries for human RAC2 (NM_002872.3 and NP 0028630.1), mouse RAC2 (NM_009008.3 and NP 033034.1), rat RAC2 (NM_001008384.1 and NP_001008385.1), chimpanzee RAC2 (XM_001145815.3 and XP_001145815.3), monkey RAC2 (XM_001086228.2 and XP_001086228.1), dog RAC2 (XM_538392.4 and XP 538392.4), cow RAC2 (NM_I 75792.2 and NP 786986.1), chicken RAC2 (NM_001201452.1 and NP 001188381.1), and zebrafish RAC2 (NM_001002061.1 and NP 001002061.1); all of which sequences (as entered by the date of filing of this application) are herein incorporated by reference. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to construct a hyperactive RAC2 protein comprising a mutation that increases activity or a recombinant nucleic acid encoding it, as described herein. In some embodiments, the RAC2 comprises a substitution of lysine for glutamic acid at amino acid position 62, wherein numbering of amino acid positions is relative to the reference amino acid sequence of SEQ ID NO:1.

Phagocytes of various embodiments in accordance with the present disclosure are naturally occurring or engineered phagocytes capable of expressing a chimeric antigen receptor, as described herein, and a hyperactive Rac GTPase and/or a Rac GTPase at an elevated expression level. Any type of phagocyte or a precursor thereof can be engineered to express a hyperactive Rac GTPase and a CAR that specifically binds to a lymphoma antigen, as described herein. Phagocytes include, without limitation, macrophages, monocytes, neutrophils, and dendritic cells. Phagocyte precursors, from which phagocytic cells may be generated, may be similarly engineered to express a hyperactive Rac GTPase and a CAR and subsequently differentiated into phagocytes. Exemplary precursor cells include bone marrow, monoblasts, hematopoietic stem cells, and other phagocyte precursor cells identifiable by a person skilled in the art. The phagocytes or phagocyte precursor cells can be autologous cells, syngeneic cells, allogeneic cells and even in some cases, xenogeneic cells.

Engineered phagocytes are targeted by the CAR receptor to lymphoma cells where, upon binding of the CAR receptor to a target lymphoma antigen, the engineered phagocytes are stimulated to perform phagocytosis or trogocytosis. For example, an engineered phagocyte may use phagocytosis to completely engulf a lymphoma cell into an internal compartment called a phagosome. Alternatively, engineered phagocytes may perform trogocytosis or partial phagocytosis, wherein the phagocyte “nibbles” at a target lymphoma cell rather than completely engulfing the target lymphoma cell. In some cases, a phagocyte may perform trogocytosis repeatedly until the target lymphoma cell disintegrates. Thus, phagocytes may kill lymphoma target cells either by phagocytosis or trogocytosis.

In addition, a suicide gene may be introduced into the engineered phagocytes, for example, to improve their safety by allowing their destruction at will. Suicide genes can be used to selectively kill cells by inducing apoptosis or converting a nontoxic drug to a toxic compound in the phagocytes. Examples include suicide genes encoding caspases, thymidine kinases, cytosine deaminases, intracellular antibodies, telomerases, and DNases. See, e.g., Jones et al. (2014) Front. Pharmacol. 5:254, Mitsui et al. (2017) Mol. Ther. Methods Clin. Dev. 5:51-58, Greco et al. (2015) Front. Pharmacol. 6:95; herein incorporated by reference. In some cases, the suicide gene is expressed from an inducible promoter to provide a “safety switch” (i.e., kill cells by inducing the suicide gene). For example, an inducible caspase-9 suicide gene system can be incorporated into engineered phagocytes as a “safety switch” (see, e.g., Straathof et al. (2005) Blood 105(11):4247-4254; Thomis et al. (2001) Blood 97(5):1249-1257; Tey et al. (2007) Biol. Blood Marrow Transplant. 13(8):913-24; herein incorporated by reference.). In some embodiments, a suicide gene is selected that expresses a human protein to minimize immune reactions in human patients treated with the engineered phagocytes.

Pharmaceutical Compositions and Cellular Therapy with Engineered Phagocytes

The engineered phagocytes that have been modified with the construct(s) encoding a CAR and/or a hyperactive Rac GTPase (e.g., RAC2^{E62K}) may be expanded in culture under selective conditions prior to administration. Pharmaceutical compositions can be prepared by formulating the engineered phagocytes, produced by the methods described herein, into dosage forms by known pharmaceutical methods. For example, a pharmaceutical composition comprising engineered phagocytes can be formulated for parenteral administration, as capsules, liquids, film-coated preparations, suspensions, emulsions, and injections (such as venous injections, drip injections, and the like).

In formulation into these dosage forms, the engineered phagocytes can be combined as appropriate, with pharmaceutically acceptable carriers or media, in particular, sterile water and physiological saline, vegetable oils, resolvents, bases, emulsifiers, suspending agents, surfactants, stabilizers, vehicles, antiseptics, binders, diluents, tonicity agents, soothing agents, bulking agents, disintegrants, buffering agents, coating agents, lubricants, coloring agents, solution adjuvants, or other additives.

The engineered phagocytes may also be used in combination with other therapeutic agents for treating lymphoma such as, but not limited to: chemotherapeutic agents such as cyclophosphamide, doxorubicin, vincristine, methotrexate, cytarabine, ifosfamide, etoposide, adriamycin, bleomycin, vinblastine, dacarbazine, chlormethine, oncovin, and procarbazine; immunotherapeutic agents such as antibodies (e.g., rituximab), cytokines (e.g., interferons, including type I (IFNα and IFNβ), type II (IFNγ) and type Ill (IFNλ) and interleukins, including interleukin-2 (IL-2)), adjuvant immunochemotherapy agents (e.g., polysaccharide-K), adoptive T-cell therapy agents, and immune checkpoint blockade therapy agents; steroids such as prednisolone, biologic therapeutic agents such as tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade); Janus kinase inhibitors, such as tofacitinib; ALK inhibitors, such as crizotinib; Bcl-2 inhibitors, such as obatoclax and gossypol; PARP inhibitors, such as Iniparib and Olaparib; PI3K inhibitors, such as perifosine; VEGF receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar); pro-apoptotic agents such as oblimersen sodium, sodium butyrate, depsipetide, fenretinide, flavipirodol, gossypol, ABT-737, ABT-263 (Navitoclax), GX15-070 and HA14-1; angiogenesis inhibitors such as bevacizumab, ramucirumab, ranibizumab, sorafenib, sunitinib, itraconazole, and carboxyamidotriazole; photoactive agents such as porfimer sodium, chlorins, bacteriochlorins, phthalocyanines, and aminolevulinic acid prodrugs; radiosensitizing agents such as cisplatin, fluoropyrimidines, gemcitabine, misonidazole, metronidazole, and taxanes; radioisotopes such as iodine-131, holmium-166, lutetium-177, radium-223, samarium-153, strontium-89, and yttrium-90; or other therapeutic agents.

In some embodiments, the pharmaceutical composition comprising the engineered phagocytes is a sustained-release formulation, or a formulation that is administered using a sustained-release device. Such devices are well known in the art, and include, for example, transdermal patches, and miniature implantable pumps that can provide for delivery of the engineered phagocytes over time in a continuous, steady-state fashion at a variety of doses to achieve a sustained-release effect with a non-sustained-release pharmaceutical composition.

Usually, but not always, the subject who receives the engineered phagocytes (i.e., the recipient) is also the subject from whom the original phagocytes (i.e., before genetic modification to express a CAR specific for a target lymphoma antigen and a hyperactive Rac) are harvested or obtained, which provides the advantage that the cells are autologous. However, phagocytes can be obtained from another subject (i.e., donor), a culture of cells from a donor, or from established cell culture lines and genetically modified, as described herein. Phagocytes may be obtained from the same or a different species than the subject to be treated, but preferably are of the same species, and more preferably of the same immunological profile as the subject. Such cells can be obtained, for example, from a biological sample comprising phagocytes from a close relative or matched donor, genetically modified to express a CAR specific for a target lymphoma antigen and a hyperactive Rac and administered to a subject in need of treatment for lymphoma. The patients or subjects who donate or receive the phagocytes are typically mammalian, and usually human. However, this need not always be the case, as veterinary applications are also contemplated. In certain embodiments, the engineered phagocytes administered to a subject are autologous or allogeneic.

At least one therapeutically effective dose of the engineered phagocytes will be administered. By “therapeutically effective dose” or “therapeutically effective amount” of the engineered phagocytes is intended an amount that when administered brings about a positive therapeutic response with respect to treatment of an individual for lymphoma. Of particular interest is an amount of the engineered phagocytes that provides an anti-tumor effect, as defined herein. By “positive therapeutic response” is intended the individual undergoing the treatment according to the invention exhibits an improvement in one or more symptoms of the lymphoma for which the individual is undergoing therapy.

Thus, for example, a “positive therapeutic response” would be an improvement in the disease in association with the therapy, and/or an improvement in one or more symptoms of the disease in association with the therapy. Therefore, for example, a positive therapeutic response would refer to one or more of the following improvements in the disease: (1) reduction in tumor size; (2) reduction in the number of lymphoma cells; (3) inhibition (i.e., slowing to some extent, preferably halting) of tumor growth; (4) inhibition (i.e., slowing to some extent, preferably halting) of cancer cell infiltration into peripheral organs; (5) inhibition (i.e., slowing to some extent, preferably halting) of tumor metastasis; and (6) some extent of relief from one or more symptoms associated with the lymphoma. Such therapeutic responses may be further characterized as to degree of improvement. Thus, for example, an improvement may be characterized as a complete response. By “complete response” is documentation of the disappearance of all symptoms and signs of all measurable or evaluable disease confirmed by physical examination, laboratory, nuclear and radiographic studies (i.e., CT (computer tomography) and/or MRI (magnetic resonance imaging)), and other non-invasive procedures repeated for all initial abnormalities or sites positive at the time of entry into the study. Alternatively, an improvement in the disease may be categorized as being a partial response. By “partial response” is intended a reduction of greater than 50% in the sum of the products of the perpendicular diameters of all measurable lesions when compared with pretreatment measurements (for patients with evaluable response only, partial response does not apply).

The engineered phagocytes described herein may be used to treat any type of lymphoma at any stage (e.g., stage I, stage II, stage Ill, or stage IV according to the Ann Arbor staging classification scheme), including, but not limited to, Hodgkin lymphoma, including classic Hodgkin lymphoma and nodular lymphocyte predominant Hodgkin lymphoma; and non-Hodgkin lymphoma, including B-cell lymphomas (e.g., Burkitt lymphoma, diffuse large B-cell lymphoma, follicular lymphoma, chronic lymphocytic leukemia, mantle cell lymphoma, marginal zone lymphoma, and lymphoplasmacytic lymphoma), T-cell lymphomas (e.g., adult T-cell lymphoma, cutaneous T-cell lymphoma, peripheral T-cell lymphoma, anaplastic large cell lymphoma, angioimmunoblastic T-cell lymphoma, adult T-cell leukemia/lymphoma, and lymphoblastic lymphoma), extranodal NK-T-cell lymphoma, gastric lymphoma, primary effusion lymphoma, splenic marginal zone lymphoma, lymphoplasmacytic lymphoma, and multicentric Castleman disease. The term lymphoma also includes any type of lymphoma associated with an infection by Epstein-Barr virus, human T-cell leukemia virus, Helicobacter pylori, human gammaherpesvirus 8, hepatitis C virus, or human immunodeficiency virus.

The pharmaceutical compositions comprising the engineered phagocytes may be administered using any route of administration in accordance with any medically acceptable method known in the art. Suitable routes of administration include parenteral administration, such as intravenous (IV), intraarterial, infusion, subcutaneous (SC), intraperitoneal (IP), intramuscular (IM), pulmonary, nasal, topical, or transdermal. In some embodiments, the pharmaceutical composition comprising the engineered phagocytes is administered locally to the site of a lymphoma tumor or metastasis.

Factors influencing the respective amount of the various compositions to be administered include, but are not limited to, the mode of administration, the frequency of administration, the particular type of lymphoma undergoing therapy, the severity of the disease, the history of the disease, whether the individual is undergoing concurrent therapy with another therapeutic agent, and the age, height, weight, health, and physical condition of the individual undergoing therapy. Generally, a higher dosage is preferred with increasing weight of the subject undergoing therapy.

In certain embodiments, multiple therapeutically effective doses of the engineered phagocytes will be administered for a time period sufficient to effect at least a partial tumor response and more preferably a complete tumor response. Where a subject undergoing cellular therapy with the engineered phagocytes exhibits a partial response, or a relapse following a prolonged period of remission, subsequent courses of cellular therapy with the engineered phagocytes may be needed to achieve complete remission of the disease. Thus, subsequent to a period of time off from a first treatment period, a subject may receive one or more additional treatment periods comprising cellular therapy with engineered phagocytes. Such a period of time off between treatment periods is referred to herein as a time period of discontinuance. It is recognized that the length of the time period of discontinuance is dependent upon the degree of tumor response (i.e., complete versus partial) achieved with any prior treatment periods of cellular therapy with the engineered phagocytes or other therapeutic agents.

### Nucleic Acids Encoding a CAR and Hyperactive Rac

In some embodiments, recombinant nucleic acids encoding a CAR and a hyperactive Rac (e.g., RAC2^{E62K}) can be introduced into phagocytes in vivo or ex vivo to treat lymphoma. Nucleic acids described herein can be inserted into an expression vector to create an expression cassette capable of producing the CAR and hyperactive Rac in a suitable host phagocyte or precursor thereof. The CAR and hyperactive Rac can be expressed from the same vector or separate vectors. The ability of constructs to produce the CAR and hyperactive Rac can be empirically determined.

Expression cassettes typically include control elements operably linked to the coding sequence, which allow for the expression of the gene in vivo in the subject species. For example, typical promoters for mammalian cell expression include the SV40 early promoter, a CMV promoter such as the CMV immediate early promoter, the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression. In some embodiments, the promoter is a phagocyte promoter that drives or regulates expression in phagocytes. Phagocyte promoters include promoters of macrophages, monocytes, neutrophils, dendritic cells, and osteoclasts. Examples of such promoters include, but are not limited to, the CSF-1, CD68, CD11c, DC-SIGN, DC-STAMP, Langerin, and human neutrophil elastase promoters, and any synthetic promoter containing elements of a phagocyte system designed to achieve high level of expression in phagocytic cells. In certain embodiments, constructs contain constitutive, conditional and/or phagocyte promoters (examples include CSF-1, CD68, p47phox promoter etc.) containing regulatory sequences for cell type specific expression (e.g., macrophage-specific transcription factor PU.1, Ets family transcription factors and STAT1, C/EBP-α, C/EBP-δ, IRF9, KLF6, and NF-κB transcription factors), DNA and RNA-binding proteins (e.g. EWS and FUS/TLS) upstream of an overexpressed or activated Rac gene. In some embodiments, the promoter is a conditional promoter, optionally wherein the conditional promoter is selected from TET (tetracycline-response elements, TET-ON/TET-OFF), Lac, dCas-transactivator, Zinc-finger-TF, TALENs-ZF Gal4-uas, synNotch and inducible promoters based on endogenous signals TNF-alpha, and cFOS promoter.

Typically, transcription termination and polyadenylation sequences will also be present, located 3′ to the translation stop codon. Preferably, a sequence for optimization of initiation of translation, located 5′ to the coding sequence, is also present. Examples of transcription terminator/polyadenylation signals include those derived from SV40, as described in Sambrook et al., supra, as well as a bovine growth hormone terminator sequence.

Enhancer elements may also be used herein to increase expression levels of the mammalian constructs. Examples include the SV40 early gene enhancer, as described in Dijkema et al., EMPO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence.

Additionally, 5′-UTR sequences can be placed adjacent to the coding sequence in order to enhance expression of the same. Such sequences may include UTRs comprising an internal ribosome entry site (IRES). Inclusion of an IRES permits the translation of one or more open reading frames from a vector to allow, for example, co-expression of the CAR and the hyperactive Rac from the same vector. The IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996) 229:295-298; Rees et al., BioTechniques (1996) 20:102-110; Kobayashi et al., BioTechniques (1996) 21:399-402; and Mosser et al., BioTechniques (1997) 22 150-161. A multitude of IRES sequences are known and include sequences derived from a wide variety of viruses, such as from leader sequences of picornaviruses such as the encephalomyocarditis virus (EMCV) UTR (Jang et al. J. Virol. (1989) 63:1651-1660), the polio leader sequence, the hepatitis A virus leader, the hepatitis C virus IRES, human rhinovirus type 2 IRES (Dobrikova et al., Proc. Natl. Acad. Sci. (2003) 100(25):15125-15130), an IRES element from the foot and mouth disease virus (Ramesh et al., Nucl. Acid Res. (1996) 24:2697-2700), a giardiavirus IRES (Garlapati et al., J. Biol. Chem. (2004) 279(5):3389-3397), and the like. A variety of nonviral IRES sequences will also find use herein, including, but not limited to IRES sequences from yeast, as well as the human angiotensin II type 1 receptor IRES (Martin et al., Mol. Cell Endocrinol. (2003) 212:51-61), fibroblast growth factor IRESs (FGF-1 IRES and FGF-2 IRES, Martineau et al. (2004) Mol. Cell. Biol. 24(17):7622-7635), vascular endothelial growth factor IRES (Baranick et al. (2008) Proc. Natl. Acad. Sci. U.S.A. 105(12):4733-4738, Stein et al. (1998) Mol. Cell. Biol. 18(6):3112-3119, Bert et al. (2006) RNA 12(6):1074-1083), and insulin-like growth factor 2 IRES (Pedersen et al. (2002) Biochem. J. 363(Pt 1):37-44). These elements are readily commercially available in plasmids sold, e.g., by Clontech (Mountain View, CA), Invivogen (San Diego, CA), Addgene (Cambridge, MA) and GeneCopoeia (Rockville, MD). See also IRESite: The database of experimentally verified IRES structures (iresite.org). An IRES sequence may be included in a vector, for example, to express multiple protein products in combination.

Alternatively, a polynucleotide encoding a viral T2A peptide can be used to allow production of multiple protein products from a single vector to allow, for example, co-expression of the CAR and the hyperactive RAC from the same vector. 2A linker peptides are inserted between the coding sequences in the multicistronic construct. The 2A peptide, which is self-cleaving, allows co-expressed proteins from the multicistronic construct to be produced at equimolar levels. 2A peptides from various viruses may be used, including, but not limited to 2A peptides derived from the foot-and-mouth disease virus, equine rhinitis A virus, Thosea asigna virus and porcine teschovirus-1. See, e.g., Kim et al. (2011) PLoS One 6(4):e18556, Trichas et al. (2008) BMC Biol. 6:40, Provost et al. (2007) Genesis 45(10):625-629, Furler et al. (2001) Gene Ther. 8(11):864-873; herein incorporated by reference in their entireties.

In certain embodiments, cells containing the construct are identified in vitro or in vivo by including a selection marker expression cassette in the construct. Selection markers confer an identifiable change to the cell permitting positive selection of cells having the construct. For example, fluorescent or bioluminescent markers (e.g., green fluorescent protein (GFP), enhanced green fluorescent protein (EGFP), Dronpa, mCherry, mOrange, mPlum, Venus, YPet, phycoerythrin, or luciferase), cell surface markers, expression of a reporter gene (e.g., GFP, dsRed, GUS, lacZ, CAT), drug selection markers such as genes that confer resistance to neomycin, puromycin, hygromycin, DHFR, GPT, zeocin, or histidinol may be used to identify cells. Alternatively, enzymes such as herpes simplex virus thymidine kinase (tk) or chloramphenicol acetyltransferase (CAT) may be employed. Any selectable marker may be used as long as it is capable of being expressed in the cell to allow identification of genetically modified cells containing the construct. Further examples of selectable markers are well known to one of skill in the art.

In certain embodiments, the selection marker expression cassette encodes two or more selection markers. Selection markers may be used in combination; for example, a cell surface marker may be used with a fluorescent marker, or a drug resistance gene may be used with a suicide gene. In certain embodiments, the selection marker expression cassette is multicistronic to allow expression of multiple selection markers in combination. The multicistronic vector may include an IRES or viral 2A peptide to allow expression of more than one selection marker from a single vector.

In certain embodiments, a suicide marker is included as a negative selection marker to facilitate negative selection of cells. Suicide genes can be used to selectively kill cells by inducing apoptosis or converting a nontoxic drug to a toxic compound in genetically modified cells. Examples include suicide genes encoding thymidine kinases, cytosine deaminases, intracellular antibodies, telomerases, caspases, and DNases. In certain embodiments, a suicide gene is used in combination with one or more other selection markers, such as those described above for use in positive selection of cells. In addition, a suicide gene may be used in genetically modified cells, for example, to improve their safety by allowing their destruction at will. See, e.g., Jones et al. (2014) Front. Pharmocol. 5:254, Mitsui et al. (2017) Mol. Ther. Methods Clin. Dev. 5:51-58, Greco et al. (2015) Front. Pharmacol. 6:95; herein incorporated by reference.

Once complete, the constructs encoding the CAR and the hyperactive Rac can be administered to a subject using standard gene delivery protocols. Methods for gene delivery are known in the art. See, e.g., U.S. Pat. Nos. 5,399,346, 5,580,859, 5,589,466. Genes can be delivered either directly to a subject or, alternatively, delivered ex vivo, to cells derived from the subject and the cells reimplanted in the subject.

A number of viral based systems have been developed for gene transfer into mammalian cells. These include adenoviruses, retroviruses (γ-retroviruses and lentiviruses), poxviruses, adeno-associated viruses, baculoviruses, and herpes simplex viruses (see e.g., Warnock et al. (2011) Methods Mol. Biol. 737:1-25; Walther et al. (2000) Drugs 60(2):249-271; and Lundstrom (2003) Trends Biotechnol. 21(3):117-122; herein incorporated by reference).

For example, retroviruses provide a convenient platform for gene delivery systems. Selected sequences can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; Boris-Lawrie and Temin (1993) Cur. Opin. Genet. Develop. 3:102-109; and Ferry et al. (2011) Curr Pharm Des. 17(24):2516-2527). Lentiviruses are a class of retroviruses that are particularly useful for delivering polynucleotides to mammalian cells because they are able to infect both dividing and nondividing cells (see e.g., Lois et al (2002) Science 295:868-872; Durand et al. (2011) Viruses 3(2):132-159; herein incorporated by reference).

A number of adenovirus vectors have also been described. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476). Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor Laboratory Press); Carter, B. J. Current Opinion in Biotechnology (1992) 3:533-539; Muzyczka, N. Current Topics in Microbiol. and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering the polynucleotides encoding a CAR and hyperactive Rac is the enterically administered recombinant poxvirus vaccines described by Small, Jr., P. A., et al. (U.S. Pat. No. 5,676,950, issued Oct. 14, 1997, herein incorporated by reference).

Additional viral vectors which will find use for delivering the nucleic acid molecules encoding the CAR and hyperactive Rac include those derived from the pox family of viruses, including vaccinia virus and avian poxvirus. By way of example, vaccinia virus recombinants expressing the CAR and hyperactive Rac can be constructed as follows. The DNA encoding the particular CAR and hyperactive Rac coding sequence is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the coding sequences of interest into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can also be used to deliver the genes. Recombinant avipox viruses, expressing immunogens from mammalian pathogens, are known to confer protective immunity when administered to non-avian species. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with. respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the Alphavirus genus, such as, but not limited to, vectors derived from the Sindbis virus (SIN), Semliki Forest virus (SFV), and Venezuelan Equine Encephalitis virus (VEE), will also find use as viral vectors for delivering the polynucleotides of the present invention. For a description of Sindbis-virus derived vectors useful for the practice of the instant methods, see, Dubensky et al. (1996) J. Virol. 70:508-519; and International Publication Nos. WO 95/07995, WO 96/17072; as well as Dubensky, Jr., T. W., et al., U.S. Pat. No. 5,843,723, issued Dec. 1, 1998, and Dubensky, Jr., T. W., U.S. Pat. No. 5,789,245, issued Aug. 4, 1998, both herein incorporated by reference. Particularly preferred are chimeric alphavirus vectors comprised of sequences derived from Sindbis virus and Venezuelan equine encephalitis virus. See, e.g., Perri et al. (2003) J. Virol. 77: 10394-10403 and International Publication Nos. WO 02/099035, WO 02/080982, WO 01/81609, and WO 00/61772; herein incorporated by reference in their entireties.

A vaccinia-based infection/transfection system can be conveniently used to provide for inducible, transient expression of the coding sequences of interest (for example, a CAR and hyperactive Rac expression cassette) in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide of interest, driven by a T7 promoter. The polymerase expressed in the cytoplasm from the vaccinia virus recombinant transcribes the transfected DNA into RNA which is then translated into protein by the host translational machinery. The method provides for high level, transient, cytoplasmic production of large quantities of RNA and its translation products. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

As an alternative approach to infection with vaccinia or avipox virus recombinants, or to the delivery of genes using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more template. Concomitantly, there will be a cDNA whose expression is under the control of the T7 promoter. Thus, some of the T7 RNA polymerase generated from translation of the amplification template RNA will lead to transcription of the desired gene. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

The synthetic expression cassette of interest can also be delivered without a viral vector. For example, the synthetic expression cassette can be packaged as DNA or RNA in liposomes prior to delivery to the subject or to cells derived therefrom. Lipid encapsulation is generally accomplished using liposomes which are able to stably bind or entrap and retain nucleic acid. The ratio of condensed DNA to lipid preparation can vary but will generally be around 1:1 (mg DNA:micromoles lipid), or more of lipid. For a review of the use of liposomes as carriers for delivery of nucleic acids, see, Hug and Sleight, Biochim. Biophys. Acta. (1991.) 1097:1-17; Straubinger et al., in Methods of Enzymology (1983), Vol. 101, pp. 512-527.

Liposomal preparations for use in the present invention include cationic (positively charged), anionic (negatively charged) and neutral preparations, with cationic liposomes particularly preferred. Cationic liposomes have been shown to mediate intracellular delivery of plasmid DNA (Felgner et al., Proc. Natl. Acad. Sci. USA (1987) 84:7413-7416); mRNA (Malone et al., Proc. Natl. Acad. Sci. USA (1989) 86:6077-6081); and purified transcription factors (Debs et al., J. Biol. Chem. (1990) 265:10189-10192), in functional form.

Cationic liposomes are readily available. For example, N[1-2,3-dioleyloxy)propyl]-N,N,N-triethylammonium (DOTMA) liposomes are available under the trademark Lipofectin, from GIBCO BRL, Grand Island, N.Y. (See, also, Felgner et al., Proc. Natl. Acad. Sci. USA (1987) 84:7413-7416). Other commercially available lipids include (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA (1978) 75:4194-4198; PCT Publication No. WO 90/11092 for a description of the synthesis of DOTAP (1,2-bis(oleoyloxy)-3-(trimethylammonio)propane) liposomes.

Similarly, anionic and neutral liposomes are readily available, such as, from Avanti Polar Lipids (Birmingham, AL), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

The liposomes can comprise multilammelar vesicles (MLVs), small unilamellar vesicles (SUVs), or large unilamellar vesicles (LUVs). The various liposome-nucleic acid complexes are prepared using methods known in the art. See, e.g., Straubinger et al., in Methods of Immunology (1983), Vol. 101, pp. 512-527; Szoka et al., Proc. Natl. Acad. Sci. USA (1978) 75:4194-4198; Papahadjopoulos et al., Biochim. Biophys. Acta (1975) 394:483; Wilson et al., Cell (1979) 17:77); Deamer and Bangham, Biochim. Biophys. Acta (1976) 443:629; Ostro et al., Biochem. Biophys. Res. Commun. (1977) 76:836; Fraley et al., Proc. Natl. Acad. Sci. USA (1979) 76:3348); Enoch and Strittmatter, Proc. Natl. Acad. Sci. USA (1979) 76:145); Fraley et al., J. Biol. Chem. (1980) 255:10431; Szoka and Papahadjopoulos, Proc. Natl. Acad. Sci. USA (1978) 75:145; and Schaefer-Ridder et al., Science (1982) 215:166.

The DNA and/or peptide(s) can also be delivered in cochleate lipid compositions similar to those described by Papahadjopoulos et al., Biochem. Biophys. Acta (1975) 394:483-491. See, also, U.S. Pat. Nos. 4,663,161 and 4,871,488.

The expression cassette of interest may also be encapsulated, adsorbed to, or associated with, particulate carriers. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; McGee J. P., et al., J Microencapsul. 14(2):197-210, 1997; O'Hagan D. T., et al., Vaccine 11(2):149-54, 1993.

Furthermore, other particulate systems and polymers can be used for the in vivo or ex vivo delivery of the nucleic acid of interest. For example, polymers such as polylysine, polyarginine, polyornithine, spermine, spermidine, as well as conjugates of these molecules, are useful for transferring a nucleic acid of interest. Similarly, DEAE dextran-mediated transfection, calcium phosphate precipitation or precipitation using other insoluble inorganic salts, such as strontium phosphate, aluminum silicates including bentonite and kaolin, chromic oxide, magnesium silicate, talc, and the like, will find use with the present methods. See, e.g., Felgner, P. L., Advanced Drug Delivery Reviews (1990) 5:163-187, for a review of delivery systems useful for gene transfer. Peptoids (Zuckerman, R. N., et al., U.S. Pat. No. 5,831,005, issued Nov. 3, 1998, herein incorporated by reference) may also be used for delivery of a construct of the present invention. Suitable transfection methods for constructs encoding a CAR are described in U.S. Patent Application Publications Nos. 20200239592 and 20200055917, and U.S. Pat. No. 10,125,193 and as well as in Morrissey et al. (2018) Elife 7:e36688; herein incorporated by reference in their entireties.

Additionally, biolistic delivery systems employing particulate carriers such as gold and tungsten, are especially useful for delivering synthetic expression cassettes encoding CAR and hyperactive Rac. The particles are coated with the synthetic expression cassette(s) to be delivered and accelerated to high velocity, generally under a reduced atmosphere, using a gun powder discharge from a “gene gun.” For a description of such techniques, and apparatuses useful therefore, see, e.g., U.S. Pat. Nos. 4,945,050; 5,036,006; 5,100,792; 5,179,022; 5,371,015; and 5,478,744. Also, needle-less injection systems can be used (Davis, H. L., et al, Vaccine 12:1503-1509, 1994; Bioject, Inc., Portland, Oreg.).

Recombinant vectors carrying a synthetic expression cassette encoding a CAR and a hyperactive Rac are formulated into compositions for delivery to a vertebrate subject. These compositions may either be prophylactic (to prevent lymphoma progression or metastasis) or therapeutic (to treat lymphoma). The compositions will comprise a “therapeutically effective amount” of the nucleic acid of interest such that an amount of the CAR and hyperactive Rac GTPase (or a biologically active fragment thereof) can be produced in vivo in phagocytes or precursors thereof to increase phagocytosis and/or trogocytosis of lymphoma cells in the individual to which it is administered. The exact amount necessary will vary depending on the subject being treated; the age and general condition of the subject to be treated; the degree of protection desired; the severity of the condition being treated; the particular CAR and hyperactive Rac protein produced and its mode of administration, among other factors. An appropriate effective amount can be readily determined by one of skill in the art. Thus, a “therapeutically effective amount” will fall in a relatively broad range that can be determined through routine trials.

The compositions will generally include one or more “pharmaceutically acceptable excipients or vehicles” such as water, saline, glycerol, polyethyleneglycol, hyaluronic acid, ethanol, etc. Additionally, auxiliary substances, such as wetting or emulsifying agents, pH buffering substances, surfactants and the like, may be present in such vehicles. Certain facilitators of nucleic acid uptake and/or expression can also be included in the compositions or coadministered.

Once formulated, the compositions can be administered directly to the subject (e.g., as described above) or, alternatively, delivered ex vivo, to cells derived from the subject, using methods such as those described above. For example, methods for the ex vivo delivery and reimplantation of transformed cells into a subject are known in the art and can include, e.g., dextran-mediated transfection, calcium phosphate precipitation, polybrene mediated transfection, lipofectamine and LT-1 mediated transfection, protoplast fusion, electroporation, encapsulation of the polynucleotide(s) in liposomes, and direct microinjection of the DNA into nuclei.

Direct delivery of synthetic expression cassette compositions in vivo will generally be accomplished with or without viral vectors, as described above, by injection using either a conventional syringe, needless devices such as Bioject™ or a gene gun, such as the Accell™ gene delivery system (PowderMed Ltd, Oxford, England).

### Kits

Also provided are kits comprising any of the compositions described herein. In certain embodiments, the kit comprises engineered phagocytes expressing a CAR specific for a target lymphoma antigen and a hyperactive Rac GTPase (e.g., hyperactive RAC2), or reagents for preparing them. For example, the kit may comprise engineered phagocytes in a pharmaceutical composition suitable for use in treatment. Alternatively, the kit may comprise one or more viral vectors or mRNA encoding the CAR specific for a target lymphoma antigen and a hyperactive Rac GTPase. In some embodiments, the hyperactive Rac GTPase is a RAC2 variant comprising a substitution of lysine for glutamic acid at amino acid position 62 (wherein numbering of amino acid positions is relative to the reference amino acid sequence of SEQ ID NO:1).

Kits may comprise one or more containers of the compositions described herein. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle). The kit can further comprise a container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery device. The kit may also provide a delivery device pre-filled with the engineered phagocytes or one or more viral vectors or mRNA encoding the CAR specific for a target lymphoma antigen and a hyperactive Rac GTPase (e.g., RAC2^{E62K}).

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, Blu-ray, flash drive, and the like, on which the information has been recorded. Yet another form of these instructions that may be present is a website address which may be used via the internet to access the information at a removed site.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

### EXAMPLES OF NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure numbered 1-86 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below. 1. A composition comprising a phagocyte specific for lymphoma cells, wherein the phagocyte is engineered to express a chimeric antigen receptor that specifically binds to a target lymphoma antigen, wherein the phagocyte is further engineered to express a hyperactive Rac GTPase.

2. The composition of aspect 1, further comprising a pharmaceutically acceptable excipient.

3. The composition of aspect 1 or 2, wherein the phagocyte is a macrophage, a monocyte, a neutrophil, or a dendritic cell, or a precursor thereof.

4. The composition of any one of aspects 1-3, wherein expression of the hyperactive Rac GTPase in the phagocyte increases phagocytic activity of the phagocyte.

5. The composition of any one of aspects 1-4, wherein the hyperactive Rac GTPase is a hyperactive Rac family small GTPase 2 (RAC2).

6. The composition of aspect 5, wherein the hyperactive RAC2 comprises a substitution of lysine for glutamic acid at amino acid position 62, wherein numbering of amino acid positions is relative to the reference amino acid sequence of SEQ ID NO:1.

7. The composition of any one of aspects 1-6, wherein the chimeric antigen receptor comprises a transmembrane domain linked to an extracellular antigen binding domain that binds specifically to the target lymphoma antigen and an intracellular engulfment signaling domain, wherein the transmembrane domain is positioned between the extracellular antigen binding domain and the intracellular engulfment signaling domain.

8. The composition of aspect 7, wherein the extracellular antigen binding domain comprises a single chain variable fragment (scFv), an antigen-binding fragment (Fab), a nanobody, a heavy chain variable (VH) domain, a light chain variable (VL) domain, a single domain antibody (sdAb), a shark variable domain of a new antigen receptor (VNAR), a single variable domain on a heavy chain (VHH), a bispecific antibody, a diabody, or a functional fragment thereof that binds specifically to the target lymphoma antigen.

9. The composition of aspect 7 or 8, wherein the transmembrane domain is a CD8, Megf10, FcRγ, Bai1, MerTK, TIM4, Stabilin-1, Stabilin-2, RAGE, CD300f, integrin subunit αv, integrin subunit β5, CD36, LRP1, SCARF1, C1Qa, Axl, CD45, or CD86 transmembrane domain.

10. The composition of any one of aspects 7-9, wherein the chimeric antigen receptor further comprises an extracellular spacer domain positioned between and connecting the extracellular antigen binding domain and the transmembrane domain.

11. The composition of any one of aspects 7-10, wherein the chimeric antigen receptor further comprises an intracellular spacer domain positioned between and connecting the intracellular engulfment signaling domain and the transmembrane domain.

12. The composition of any one of aspects 1-11, wherein the target lymphoma antigen is selected from the group consisting of CD2, CD3, CD4, CD5, CD10, CD15, CD19, CD20, CD22, CD30, CD43, CD79a, BCL-2, BCL6, Pax-5, TdT, LCA, Oct-2, BOB.1, Ki67, and Epstein-Barr virus-latent membrane protein (EBV-LMP).

13. The composition of aspect 12, wherein the target lymphoma antigen is CD19.

14. The composition of any one of aspects 1-13, wherein the lymphoma is Burkitt lymphoma.

15. The composition of any one of aspects 1-14, further comprising a lymphoma cell.

16. The composition of aspect 15, wherein the phagocyte is associated with all or a portion of the lymphoma cell, wherein the chimeric antigen receptor specifically binds to the target lymphoma antigen on the lymphoma cell.

17. The composition of aspect 15 or 16, wherein the lymphoma cell is partially or fully engulfed by the engineered phagocyte.

18. The composition of any one of aspects 1-17, wherein the hyperactive Rac GTPase is expressed as a fusion protein linked to the chimeric antigen receptor by a cleavable peptide.

19. The composition of any one of aspects 1-18, wherein the hyperactive Rac GTPase is expressed independently of the chimeric antigen receptor.

20. The composition of any one of aspects 1-19, wherein the phagocyte is engineered to express the chimeric antigen receptor and the hyperactive Rac GTPase by transfecting the phagocyte with a first recombinant polynucleotide encoding the chimeric antigen receptor and a second recombinant polynucleotide encoding the hyperactive Rac GTPase or transfecting the phagocyte with a bicistronic recombinant polynucleotide encoding the chimeric antigen receptor and the hyperactive Rac GTPase.

21. The composition of aspect 20, wherein the first recombinant polynucleotide, the second recombinant polynucleotide, or the bicistronic recombinant polynucleotide is a plasmid, a viral vector, or a messenger RNA.

22. The composition of aspect 20 or 21, wherein the bicistronic recombinant polynucleotide comprises an internal ribosome entry site (IRES) or a 2A element.

23. The composition of any one of aspects 20-22, wherein the first recombinant polynucleotide comprises a promoter operably linked to a nucleotide sequence encoding the chimeric antigen receptor.

24. The composition of any one of aspects 20-23, wherein the second recombinant polynucleotide comprises a promoter operably linked to a nucleotide sequence encoding the hyperactive Rac GTPase.

25. The composition of aspect 24, wherein the second recombinant polynucleotide further comprises a nucleotide sequence encoding the chimeric antigen receptor.

26. The composition of any one of aspects 23-25, wherein the promoter is constitutive or inducible.

27. The composition of any one of aspects 23-26, wherein the promoter is a phagocyte promoter.

28. The composition of any one of aspects 1-27, further comprising an anti-cancer therapeutic agent.

29. The composition of aspect 28, wherein the anti-cancer therapeutic agent is selected from the group consisting of a chemotherapeutic agent, an immunotherapeutic agent, a biologic therapeutic agent, a pro-apoptotic agent, an angiogenesis inhibitor, a photoactive agent, a radiosensitizing agent, and a radioisotope.

30. A method of treating lymphoma in a subject in need thereof, the method comprising administering to the subject a therapeutically effective amount of the composition of any one of aspects 1-29.

31. The method of aspect 30, wherein the phagocyte has anti-tumor activity.

32. The method of aspect 30 or 31, wherein expression of the hyperactive Rac GTPase is inducible.

33. The method of any one of aspects 30-32, wherein the phagocyte is autologous or allogeneic.

34. The method of any one of aspects 30-33, wherein the phagocyte is engineered in vitro or ex vivo and expanded in culture prior to said administering.

35. The method of any one of aspects 30-34, wherein the subject is a human.

36. The method of any one of aspects 30-35, further comprising administering an anti-cancer therapeutic agent.

37. The method of aspect 36, wherein the anti-cancer therapeutic agent is selected from the group consisting of a chemotherapeutic agent, an immunotherapeutic agent, a biologic therapeutic agent, a pro-apoptotic agent, an angiogenesis inhibitor, a photoactive agent, a radiosensitizing agent, and a radioisotope.

38. The method of aspect 37, wherein the chemotherapeutic agent is selected from the group consisting of methotrexate, cyclophosphamide, doxorubicin, vincristine, cytarabine, ifosfamide, and etoposide.

39. The method of aspect 37, wherein the immunotherapeutic agent is rituximab.

40. The method of any one of aspects 30-39, further comprising administering a steroid.

41. The method of aspect 40, wherein the steroid is prednisolone.

42. The method of any one of aspects 30-41, wherein multiple therapeutically effective doses of the phagocyte are administered to said subject.

43. The method of aspect 42, wherein multiple cycles of treatment are administered to said subject for a time period sufficient to effect at least a partial tumor response.

44. The method of aspect 43, wherein the time period is at least 6 months.

45. The method of aspect 44, wherein the time period is at least 12 months.

46. The method of any one of aspects 43-45, wherein a complete tumor response is effected.

47. The method of any one of aspects 43-46, wherein treatment results in a reduction in tumor size, a reduction in the number of lymphoma cells, slowing or halting of tumor growth, slowing or halting of cancer cell infiltration into peripheral organs, slowing or halting of tumor metastasis, or a combination thereof.

48. The method of any one of aspects 30-47, wherein the lymphoma is Burkitt lymphoma.

49. A kit comprising the composition of any one of aspects 1-29 and instructions for treating lymphoma.

50. A method of increasing phagocytosis or trogocytosis of a lymphoma cell in a subject, the method comprising administering to the subject an effective amount of an engineered phagocyte, wherein the engineered phagocyte is engineered to express a chimeric antigen receptor that specifically binds to a target lymphoma antigen on the lymphoma cell, wherein the engineered phagocyte is further engineered to express a hyperactive Rac GTPase, wherein the lymphoma cell undergoes phagocytosis or trogocytosis by the engineered phagocyte at an increased rate compared to the rate of phagocytosis or trogocytosis of a control phagocyte that is not engineered to express the chimeric antigen receptor and the hyperactive Rac GTPase.

51. The method of aspect 50, wherein the lymphoma cell is wholly or partially engulfed by the engineered phagocyte.

52. A composition comprising one or more expression vectors comprising a first expression cassette comprising a coding sequence encoding a chimeric antigen receptor that specifically binds to a target lymphoma antigen and a second expression cassette comprising a coding sequence encoding a hyperactive RAC2 GTPase, wherein the chimeric antigen receptor and the hyperactive RAC2 GTPase are expressed in vivo in a phagocyte in the subject in a therapeutically effective amount sufficient to result in phagocytosis or trogocytosis of a lymphoma cell.

53. The composition of aspect 52, wherein the hyperactive RAC2 GTPase comprises a substitution of lysine for glutamic acid at amino acid position 62, wherein numbering of amino acid positions is relative to the reference amino acid sequence of SEQ ID NO:1.

54. The composition of aspect 52 or 53, wherein the chimeric antigen receptor comprises a transmembrane domain linked to an extracellular antigen binding domain that binds specifically to the target lymphoma antigen and an intracellular engulfment signaling domain, wherein the transmembrane domain is positioned between the extracellular antigen binding domain and the intracellular engulfment signaling domain.

55. The composition of aspect 44, wherein the extracellular antigen binding domain comprises a single chain variable fragment (scFv), an antigen-binding fragment (Fab), a nanobody, a heavy chain variable (VH) domain, a light chain variable (VL) domain, a single domain antibody (sdAb), a shark variable domain of a new antigen receptor (VNAR), a single variable domain on a heavy chain (VHH), a bispecific antibody, a diabody, or a functional fragment thereof that binds specifically to the target lymphoma antigen.

56. The composition of aspect 54 or 55, wherein the transmembrane domain is a CD8, Megf10, FcRγ, Bai1, MerTK, TIM4, Stabilin-1, Stabilin-2, RAGE, CD300f, integrin subunit αv, integrin subunit β5, CD36, LRP1, SCARF1, C1Qa, Axl, CD45, or CD86 transmembrane domain.

57. The composition of any one of aspects 54-56, wherein the chimeric antigen receptor further comprises an extracellular spacer domain positioned between and connecting the extracellular antigen binding domain and the transmembrane domain.

58. The composition of any one of aspects 54-57, wherein the chimeric antigen receptor further comprises an intracellular spacer domain positioned between and connecting the intracellular engulfment signaling domain and the transmembrane domain.

59. The composition of any one of aspects 52-58, wherein the target lymphoma antigen is selected from the group consisting of CD2, CD3, CD4, CD5, CD10, CD15, CD19, CD20, CD22, CD30, CD43, CD79a, BCL-2, BCL6, Pax-5, TdT, LCA, Oct-2, BOB.1, Ki67, and Epstein-Barr virus-latent membrane protein (EBV-LMP).

60. The composition of any one of aspects 52-59, wherein the first expression cassette and the second expression cassette are in separate vectors.

61. The composition of any one of aspects 52-59, wherein the first expression cassette and the second expression cassette are in a bicistronic vector comprising an internal ribosome entry site (IRES) or a 2A element.

62. The composition of any one of aspects 52-61, wherein the first expression cassette comprises a promoter operably linked to the coding sequence encoding the chimeric antigen receptor.

63. The composition of aspect 62, wherein the promoter is a constitutive or inducible promoter.

64. The composition of aspect 62 or 63, wherein the promoter is a phagocyte promoter.

65. The composition of any one of aspects 52-64, wherein the second expression cassette comprises a promoter operably linked to the coding sequence encoding the hyperactive RAC2 GTPase.

66. The composition of aspect 65, wherein the promoter is a constitutive or inducible promoter.

67. The composition of aspect 65 or 66, wherein the promoter is a phagocyte promoter.

68. A method of treating lymphoma in a subject in need thereof, the method comprising administering one or more expression vectors comprising a first expression cassette comprising a coding sequence encoding a chimeric antigen receptor that specifically binds to a target lymphoma antigen and a second expression cassette comprising a coding sequence encoding a hyperactive RAC2 GTPase, wherein the chimeric antigen receptor and the hyperactive RAC2 GTPase are expressed in vivo in a phagocyte in the subject in a therapeutically effective amount sufficient to result in phagocytosis or trogocytosis of a lymphoma cell.

69. The method of aspect 68, wherein the hyperactive RAC2 GTPase comprises a substitution of lysine for glutamic acid at amino acid position 62, wherein numbering of amino acid positions is relative to the reference amino acid sequence of SEQ ID NO:1.

70. The method of aspect 68 or 69, wherein the chimeric antigen receptor comprises a transmembrane domain linked to an extracellular antigen binding domain that binds specifically to the target lymphoma antigen and an intracellular engulfment signaling domain, wherein the transmembrane domain is positioned between the extracellular antigen binding domain and the intracellular engulfment signaling domain.

71. The method of aspect 70, wherein the extracellular antigen binding domain comprises a single chain variable fragment (scFv), an antigen-binding fragment (Fab), a nanobody, a heavy chain variable (VH) domain, a light chain variable (VL) domain, a single domain antibody (sdAb), a shark variable domain of a new antigen receptor (VNAR), a single variable domain on a heavy chain (VHH), a bispecific antibody, a diabody, or a functional fragment thereof that binds specifically to the target lymphoma antigen.

72. The method of aspect 70 or 71, wherein the transmembrane domain is a CD8, Megf10, FcRγ, Bai1, MerTK, TIM4, Stabilin-1, Stabilin-2, RAGE, CD300f, integrin subunit αv, integrin subunit β5, CD36, LRP1, SCARF1, C1Qa, Axl, CD45, or CD86 transmembrane domain.

73. The method of any one of aspects 70-72, wherein the chimeric antigen receptor further comprises an extracellular spacer domain positioned between and connecting the extracellular antigen binding domain and the transmembrane domain.

74. The method of any one of aspects 70-73, wherein the chimeric antigen receptor further comprises an intracellular spacer domain positioned between and connecting the intracellular engulfment signaling domain and the transmembrane domain.

75. The method of any one of aspects 68-74, wherein the target lymphoma antigen is selected from the group consisting of CD2, CD3, CD4, CD5, CD10, CD15, CD19, CD20, CD22, CD30, CD43, CD79a, BCL-2, BCL6, Pax-5, TdT, LCA, Oct-2, BOB.1, Ki67, and Epstein-Barr virus-latent membrane protein (EBV-LMP).

76. The method of any one of aspects 68-75, wherein the first expression cassette and the second expression cassette are in separate vectors.

77. The method of any one of aspects 68-75, wherein the first expression cassette and the second expression cassette are in a bicistronic vector comprising an internal ribosome entry site (IRES) or a 2A element.

78. The method of any one of aspects 68-77, wherein the first expression cassette comprises a promoter operably linked to the coding sequence encoding the chimeric antigen receptor.

79. The method of aspect 78, wherein the promoter is a constitutive or inducible promoter.

80. The method of aspect 78 or 79, wherein the promoter is a phagocyte promoter.

81. The method of any one of aspects 68-80, wherein the second expression cassette comprises a promoter operably linked to the coding sequence encoding the hyperactive RAC2 GTPase.

82. The method of aspect 81, wherein the promoter is a constitutive or inducible promoter.

83. The method of aspect 81 or 82, wherein the promoter is a phagocyte promoter.

84. The method of any one of aspects 68-83, wherein the lymphoma is Burkitt lymphoma.

85. A composition comprising a phagocyte for use in a method of treating lymphoma in a subject, wherein the phagocyte is engineered to express a chimeric antigen receptor that specifically binds to a target lymphoma antigen, wherein the phagocyte is further engineered to express a hyperactive Rac GTPase, wherein the phagocyte engulfs lymphoma cells expressing the target lymphoma antigen.

86. A composition comprising one or more expression vectors comprising a first expression cassette comprising a coding sequence encoding a chimeric antigen receptor that specifically binds to a target lymphoma antigen and a second expression cassette comprising a coding sequence encoding a hyperactive RAC2 GTPase for use in a method of treating lymphoma, wherein the chimeric antigen receptor and the hyperactive RAC2 GTPase are expressed in vivo in a phagocyte in the subject in a therapeutically effective amount sufficient to result in phagocytosis or trogocytosis of a lymphoma cell.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

CAR-P Immunotherapy with Phagocytes

A first-in-human CAR-P (also known as CAR-M) clinical trial is in progress for the treatment of breast cancer, and the approach is safe (1). However, the therapy is not yet as effective as it needs to be, at least in part because CAR macrophages tend to nibble on tumor cells rather than engulfing them whole and killing them (2). Thus, enhancing whole cell engulfment is imperative.

We made a discovery in fruit flies that offers to overcome the most significant limitation to CAR-P. We have demonstrated a proof-of-concept using primary mouse cells and human cell lines. Here we propose to leverage this discovery to dramatically improve CAR-P and target the most challenging cancers. In addition, we propose to radically improve the engineering of macrophages to make it far simpler and more affordable. If successful, this innovative combination of approaches would make cellular therapies generalizable to the treatment of many more diseases including multidrug resistant bacteria, viral infections, autoimmune diseases, and more. The Microbiology Society predicts that antibiotic resistant bacteria alone will kill 10 million people/yr by 2050 without new interventions. So, the potential impact of the proposed approach is enormous.

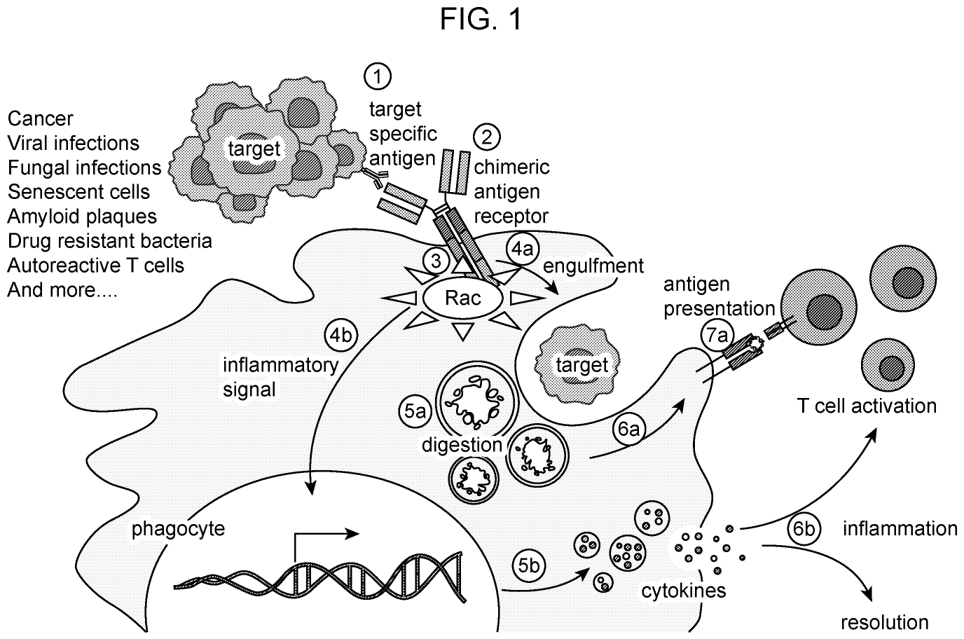

Working in Drosophila, we recently made the surprising discovery that expressing an active form of the small GTPase Rac is sufficient to drive cells to engulf and kill other living cells (FIG. 1). Here we propose to harness this effect to engineer highly effective CAR-P cells to treat the most challenging cancers. Moreover, we propose to generalize the approach to remove any undesirable cell or material from the body.

A key challenge with CAR-P is to increase the phagocytic activity of CAR macrophages, especially their ability to engulf and kill whole, living cells. Our work in Drosophila led us to a simple and remarkable solution to this challenge. Coexpression of an active form of Rac, which is a key node in the signaling and cytoskeletal networks that govern diverse cell behaviors, together with a CAR causes macrophages to avidly and specifically engulf whole living target cells of our choosing. We call this approach Rac-enhanced CAR-Phagocyte (Race CAR-P) therapy (FIG. 1). A further potential advantage of Race CAR-P over CAR-T therapy is that phagocytes can remove substances that evade T cells like antibiotic resistant bacteria, senescent cells, and auto-immune T cells, potentially greatly expanding the impact of cellular therapies. However, a significant barrier to all cellular therapies is the prohibitive cost: CAR-T is currently estimated at ˜$1M per patient including $475K for the cells and $500K to manage side effects. The approach is costly because of the complexity of removing cells from the body, engineering them ex vivo, and returning them to the patient.

Rac is sufficient to render cells hyper phagocytic. Hsu et al (29) identified human patients with an activating mutation in RAC2 (RAC2^{E62K/+}) and reported that they exhibit unexplained lymphopenia (absence of circulating B and T cells). RAC2 is one of three human Rac proteins and is expressed specifically in hematopoietic cells. RAC2^{E62K} causes a two-fold increase in RAC2 activity, mildly impairs neutrophil chemotaxis, and hyperactivates superoxide production, consistent with known functions of neutrophil RAC2. A RAC2^{E62K/+} knock-in mouse recapitulated the human phenotypes and further revealed no defect in B or T cell development, leaving the lymphopenia unexplained.

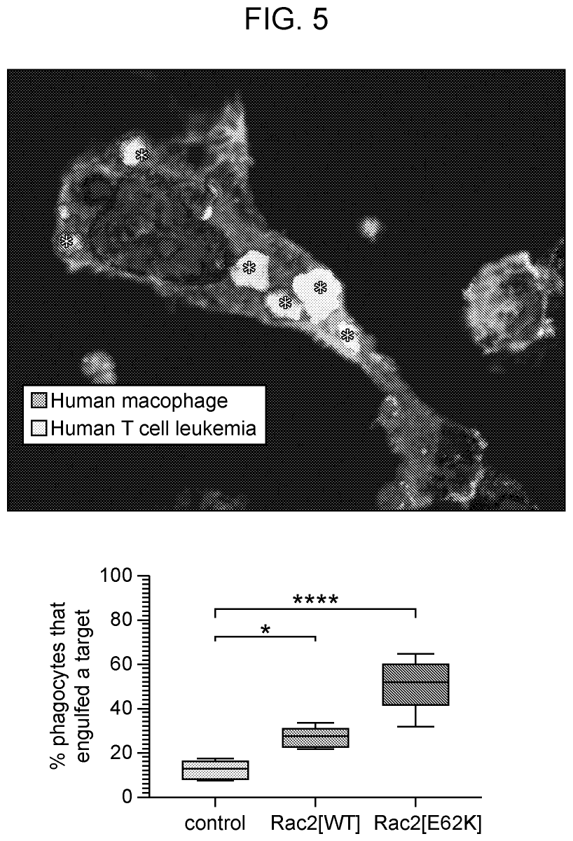

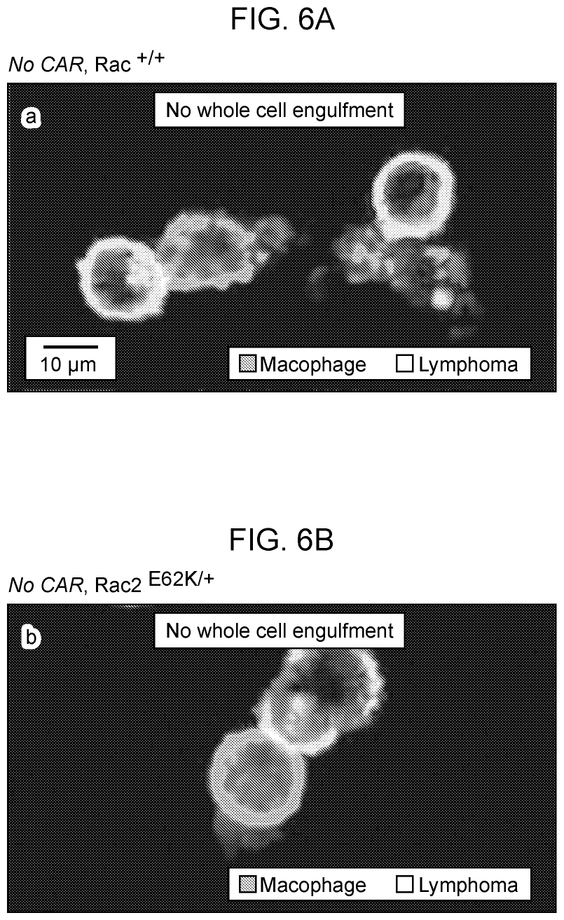

The lymphopenia was perplexing because RAC2 is required for B and T cell development, survival, and activation. It was unclear how a small increase in RAC2 activity would cause B or T cell loss. Macrophages normally engulf activated lymphocytes to resolve inflammatory responses. So, based on our findings in flies, we hypothesized that the patients' hyperactive phagocytes might be prematurely engulfing and killing their lymphocytes just as Drosophila border cells prematurely engulf and kill nurse cells. To test this idea, we isolated bone marrow derived macrophages (BMDMs) from RAC2^{E62K/+} mice and co-cultured them with T cells. As we predicted, RAC2^{E62K} macrophages avidly engulfed and killed T cells (FIG. 4). We have also over-expressed RAC2^{E62K} in human macrophages and demonstrated engulfment of human T cell leukemia cells (FIG. 5).

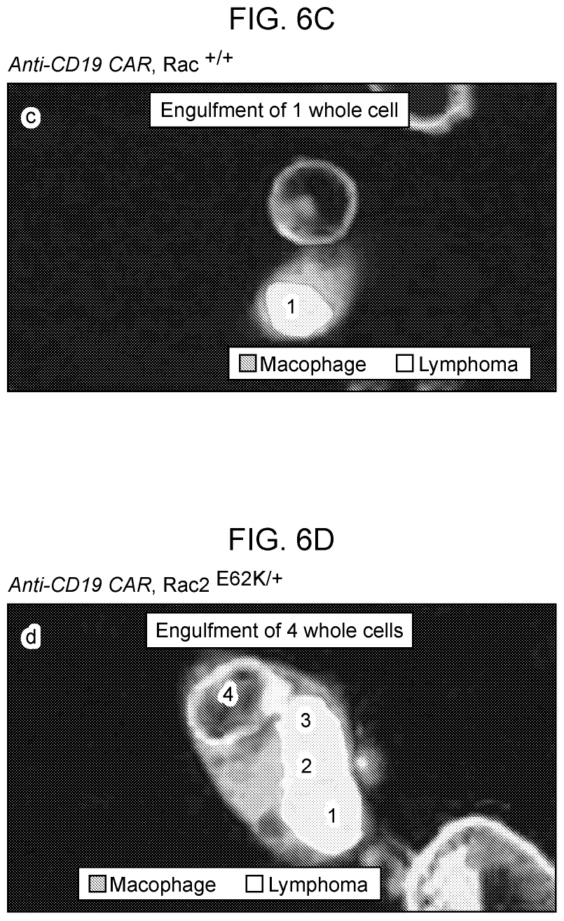

We then showed that RAC2^{E62K/+} BMDMs expressing an anti-CD19 CAR specifically engulf and kill CD19-expressing lymphoma cells (FIG. 6). Importantly, RAC2^{E62K} has no effect in the absence of the CAR (FIGS. 6A, 6B) but dramatically enhances the anti-CD19 CAR effect (FIGS. 6C, 6D).

The sufficiency of hyperactive Rac to drive whole cell engulfment was surprising because 1) the engulfment machinery is complex so there was no way to anticipate that Rac was a particularly critical signal and 2) a simple prediction would have been that hyperactive Rac would paralyze cells by over-polymerizing actin. However, in both fly and human cells, it is important to note that there are multiple Rac proteins expressed, and RAC2^{E62K} is heterozygous, so F-actin dynamics can still occur via the wild type Rac proteins. Even more surprising—in fact impossible to anticipate—was that studies of Drosophila border cells could lead to insights into immunology.

Thus, the solution to a 25-year-old cold case in Drosophila led to an explanation for an otherwise mysterious human immunodeficiency and a novel approach to CAR-P cancer immunotherapy, Race CAR-P. These results suggest that engineering macrophages to express specific receptors together with active Rac should be generally useful to remove unwanted cells or substances from the body.

### Example 2

### CAR Constructs

We engineer CARs composed of a specific, single chain antibody against the target of interest fused to a CD8 transmembrane domain, and intracellular costimulatory domains. We express them in phagocytes together with RAC2^{E62K} and test for their ability to engulf and destroy corresponding targets ex vivo. In principle, Race CAR-P has the potential to eliminate any unwanted cell or substance from the body.

### BIBLIOGRAPHY

1. C. Sloas, S. Gill, M. Klichinsky, Engineered CAR-Macrophages as Adoptive Immunotherapies for Solid Tumors. Front. Immunol. 12, 783305 (2021).
2. M. A. Morrissey et al., Chimeric antigen receptors that trigger phagocytosis. Elife. 7 (2018), doi:10.7554/eLife.36688.
3. S. B. Weisser, N. van Rooijen, L. M. Sly, Depletion and reconstitution of macrophages in mice. J. Vis. Exp., 4105 (2012).
4. H. L. Tang et al., Cell survival, DNA damage, and oncogenic transformation after a transient and reversible apoptotic response. Mol. Biol. Cell. 23, 2240-2252 (2012).
5. G. Sun et al., A molecular signature for anastasis, recovery from the brink of apoptotic cell death. J. Cell Biol. 216, 3355-3368 (2017).
6. A. X. Ding et al., CasExpress reveals widespread and diverse patterns of cell survival of caspase-3 activation during development in vivo. Elife. 5 (2016), doi:10.7554/eLife.10936.
7. G. Sun, X. A. Ding, Y. Argaw, X. Guo, D. J. Montell, Akt1 and dCIZ1 promote cell survival from apoptotic caspase activation during regeneration and oncogenic overgrowth. Nat. Commun. 11, 5726 (2020).
8. R. Mirzayans, D. Murray, Intratumor heterogeneity and therapy resistance: contributions of dormancy, apoptosis reversal (anastasis) and cell fusion to disease recurrence. Int. J. Mol. Sci. 21 (2020), doi:10.3390/ijms21041308.
9. J. Nagel, G. Sun, V. Balasanyan, D. Montell, S. Dedhar, Increased metastatic potential and multidrug resistance in triple negative breast cancer cells that undergo anastasis following chemotherapy (2019).
10. A. Conod, M. Silvano, A. Ruiz I Altaba, On the origin of metastases: Induction of pro-metastatic states after impending cell death via ER stress, reprogramming, and a cytokine storm. Cell Rep. 38, 110490 (2022).
11. A. Shemorry et al., Caspase-mediated cleavage of IRE1 controls apoptotic cell commitment during endoplasmic reticulum stress. Elife. 8 (2019), doi:10.7554/eLife.47084.
12. L. Wang et al., Anastasis Drives Senescence and Non-Cell Autonomous Neurodegeneration in the Astrogliopathy Alexander Disease. J. Neurosci. 42, 2584-2597 (2022).
13. E. Scarpa, R. Mayor, Collective cell migration in development. J. Cell Biol. 212, 143-155 (2016).
14. H. M. Olson, A. V. Nechiporuk, Using zebrafish to study collective cell migration in development and disease. Front. Cell Dev. Biol. 6, 83 (2018).
15. E. Theveneau, R. Mayor, Collective cell migration of epithelial and mesenchymal cells. Cell Mol. Life Sci. 70, 3481-3492 (2013).
16. C. J. Weijer, Collective cell migration in development. J. Cell Sci. 122, 3215-3223 (2009).
17. A. K. Mishra, J. P. Campanale, J. A. Mondo, D. J. Montell, Cell interactions in collective cell migration. Development. 146 (2019), doi:10.1242/dev.172056.
18. E. Theveneau, R. Mayor, Collective cell migration of the cephalic neural crest: the art of integrating information. Genesis. 49, 164-176 (2011).
19. A. Aman, T. Piotrowski, Cell migration during morphogenesis. Dev. Biol. 341, 20-33 (2010).
20. N. Aceto et al., Circulating tumor cell clusters are oligoclonal precursors of breast cancer metastasis. Cell. 158, 1110-1122 (2014).
21. K. J. Cheung et al., Polyclonal breast cancer metastases arise from collective dissemination of keratin 14-expressing tumor cell clusters. Proc. Natl. Acad. Sci. USA. 113, E854-63 (2016).
22. S. Y. Kok et al., Malignant subclone drives metastasis of genetically and phenotypically heterogenous cell clusters through fibrotic niche generation. Nat. Commun. 12, 863 (2021).
23. N. R. Campbell et al., Cooperation between melanoma cell states promotes metastasis through heterotypic cluster formation. Dev. Cell. 56, 2808-2825.e10 (2021).
24. A. J. Ridley, Rho GTPase signalling in cell migration. Curr. Opin. Cell Biol. 36, 103-112 (2015).
25. A. M. Murphy, D. J. Montell, Cell type-specific roles for Cdc42, Rac, and RhoL in Drosophila oogenesis. J. Cell Biol. 133, 617-630 (1996).
26. X. Wang, L. He, Y. I. Wu, K. M. Hahn, D. J. Montell, Light-mediated activation reveals a key role for Rac in collective guidance of cell movement in vivo. Nat. Cell Biol. 12, 591-597 (2010).
27. G. C. Brown, J. J. Neher, Eaten alive! Cell death by primary phagocytosis: “phagoptosis”. Trends Biochem. Sci. 37, 325-332 (2012).
28. A. D. Hoppe, J. A. Swanson, Cdc42, Rac1, and RAC2 display distinct patterns of activation during phagocytosis. Mol. Biol. Cell. 15, 3509-3519 (2004).
29. A. P. Hsu et al., Dominant activating RAC2 mutation with lymphopenia, immunodeficiency, and cytoskeletal defects. Blood. 133, 1977-1988 (2019).

1. A composition comprising a phagocyte specific for lymphoma cells, wherein the phagocyte is engineered to express a chimeric antigen receptor that specifically binds to a target lymphoma antigen, wherein the phagocyte is further engineered to express a hyperactive Rac GTPase.

2. (canceled)

3. The composition of claim 1, wherein the phagocyte is a macrophage, a monocyte, a neutrophil, or a dendritic cell, or a precursor thereof.

4. The composition of claim 1, wherein expression of the hyperactive Rac GTPase in the phagocyte increases phagocytic activity of the phagocyte.

5. The composition of claim 1, wherein the hyperactive Rac GTPase is a hyperactive Rac family small GTPase 2 (RAC2).

6. The composition of claim 5, wherein the hyperactive RAC2 comprises a substitution of lysine for glutamic acid at amino acid position 62, wherein numbering of amino acid positions is relative to the reference amino acid sequence of SEQ ID NO:1.

7. The composition of claim 1, wherein the chimeric antigen receptor comprises a transmembrane domain linked to an extracellular antigen binding domain that binds specifically to the target lymphoma antigen and an intracellular engulfment signaling domain, wherein the transmembrane domain is positioned between the extracellular antigen binding domain and the intracellular engulfment signaling domain.

8. The composition of claim 7, wherein the extracellular antigen binding domain comprises a single chain variable fragment (scFv), an antigen-binding fragment (Fab), a nanobody, a heavy chain variable (VH) domain, a light chain variable (VL) domain, a single domain antibody (sdAb), a shark variable domain of a new antigen receptor (VNAR), a single variable domain on a heavy chain (VHH), a bispecific antibody, a diabody, or a functional fragment thereof that binds specifically to the target lymphoma antigen.

9. The composition of claim 7, wherein the transmembrane domain is a CD8, Megf10, FcRγ, Bai1, MerTK, TIM4, Stabilin-1, Stabilin-2, RAGE, CD300f, integrin subunit αv, integrin subunit β5, CD36, LRP1, SCARF1, C1Qa, Axl, CD45, or CD86 transmembrane domain.

10. The composition of claim 7, wherein the chimeric antigen receptor further comprises an extracellular spacer domain positioned between and connecting the extracellular antigen binding domain and the transmembrane domain.

11. The composition of claim 7, wherein the chimeric antigen receptor further comprises an intracellular spacer domain positioned between and connecting the intracellular engulfment signaling domain and the transmembrane domain.

12. The composition of claim 1, wherein the target lymphoma antigen is selected from the group consisting of CD2, CD3, CD4, CD5, CD10, CD15, CD19, CD20, CD22, CD30, CD43, CD79a, BCL-2, BCL6, Pax-5, TdT, LCA, Oct-2, BOB.1, Ki67, and Epstein-Barr virus-latent membrane protein (EBV-LMP).

13. (canceled)

14. (canceled)

15. The composition of claim 1, further comprising a lymphoma cell.

16. The composition of claim 15, wherein the phagocyte is associated with all or a portion of the lymphoma cell, wherein the chimeric antigen receptor specifically binds to the target lymphoma antigen on the lymphoma cell.

17. The composition of claim 15, wherein the lymphoma cell is partially or fully engulfed by the engineered phagocyte.

18. The composition of claim 1, wherein the hyperactive Rac GTPase is expressed as a fusion protein linked to the chimeric antigen receptor by a cleavable peptide.

19. The composition of claim 1, wherein the hyperactive Rac GTPase is expressed independently of the chimeric antigen receptor.

20. The composition of claim 1, wherein the phagocyte is engineered to express the chimeric antigen receptor and the hyperactive Rac GTPase by transfecting the phagocyte with a first recombinant polynucleotide encoding the chimeric antigen receptor and a second recombinant polynucleotide encoding the hyperactive Rac GTPase or transfecting the phagocyte with a bicistronic recombinant polynucleotide encoding the chimeric antigen receptor and the hyperactive Rac GTPase.

21. The composition of claim 20, wherein the first recombinant polynucleotide, the second recombinant polynucleotide, or the bicistronic recombinant polynucleotide is a plasmid, a viral vector, or a messenger RNA.

22. The composition of claim 20, wherein the bicistronic recombinant polynucleotide comprises an internal ribosome entry site (IRES) or a 2A element.

23. The composition of claim 20, wherein the first recombinant polynucleotide comprises a promoter operably linked to a nucleotide sequence encoding the chimeric antigen receptor.

24. The composition of claim 20, wherein the second recombinant polynucleotide comprises a promoter operably linked to a nucleotide sequence encoding the hyperactive Rac GTPase.

25. The composition of claim 24, wherein the second recombinant polynucleotide further comprises a nucleotide sequence encoding the chimeric antigen receptor.

26. (canceled)

27. (canceled)

28. (canceled)

29. The composition of claim 1, further comprising an anti-cancer therapeutic agent is selected from the group consisting of a chemotherapeutic agent, an immunotherapeutic agent, a biologic therapeutic agent, a pro-apoptotic agent, an angiogenesis inhibitor, a photoactive agent, a radiosensitizing agent, and a radioisotope.

30.-86. (canceled)


SEQUENCE LISTING (19 sequences)

SEQ ID NO: 1 - protein, 192 aa, Homo sapiens
MQAIKCVVVGDGAVGKTCLLISYTTNAFPGEYIPTVFDNYSANVMVDSKPVNLGLWDTAGQEDYDRLRPLSYPQTDVFLICFSLVSPASYENVRAKWFPEVRHHCPSTPIILVGTKLDLRDDKDTIEKLKEKKLAPITYPQGLALAKEIDSVKYLECSALTQRGLKTVFDEAIRAVLCPQPTRQQKRACSLL

SEQ ID NO: 2 - protein, 7 aa, synthetic construct
SGGGGGS

SEQ ID NO: 3 - protein, 9 aa, synthetic construct
SGGGGGGGS

SEQ ID NO: 4 - protein, 11 aa, synthetic construct
SGGGGGGGGGS

SEQ ID NO: 5 - protein, 13 aa, synthetic construct
SGGGGGGGGGGGS

SEQ ID NO: 6 - protein, 7 aa, synthetic construct
SGGGGGG

SEQ ID NO: 7 - protein, 5 aa, synthetic construct
GGGGS

SEQ ID NO: 8 - protein, 5 aa, synthetic construct
SGGGG

SEQ ID NO: 9 - protein, 10 aa, synthetic construct
  REPEAT 1..5
GGGXXGGGGS

SEQ ID NO: 10 - protein, 10 aa, synthetic construct
  REPEAT 6..10
GGGGSXGGGS

SEQ ID NO: 11 - protein, 10 aa, synthetic construct
  REPEAT 1..5
GGGPSGGGGS

SEQ ID NO: 12 - protein, 10 aa, synthetic construct
  REPEAT 1..5
GGGGQGGGGS

SEQ ID NO: 13 - protein, 10 aa, synthetic construct
  REPEAT 1..5
GGGGAGGGGS

SEQ ID NO: 14 - protein, 10 aa, synthetic construct
  REPEAT 6..10
GGGGSPGGGS

SEQ ID NO: 15 - protein, 15 aa, synthetic construct
GGGGAGGGGAGGGGS

SEQ ID NO: 16 - protein, 15 aa, synthetic construct
GGGGQGGGGQGGGGS

SEQ ID NO: 17 - protein, 15 aa, synthetic construct
GGGPSGGGPSGGGGS

SEQ ID NO: 18 - protein, 5 aa, synthetic construct
GSGGS

SEQ ID NO: 19 - protein, 6 aa, synthetic construct
SGGSGS